(19) United States
(12) Patent Application Publication
(10) Pub. No.: US 2022/0082572 A1
(43) Pub. Date: Mar. 17, 2022

(54) USE OF PERIPHERAL BLOOD PRO- AND ANTI-INFLAMMATORY CYTOKINE RATIOS AS MARKERS OF IMPLANTATION PRECEDING PREGNANCY

(71) Applicant: The Chinese University of Hong Kong, Hong Kong (CN)

(72) Inventors: Tin Chiu LI, Hong Kong (CN); Chi Chiu WANG, Hong Kong (CN); Tao ZHANG, Hong Kong (CN); Yiwei ZHAO, Hong Kong (CN); Xiaoyan CHEN, Hong Kong (CN)

(21) Appl. No.: 17/024,206

(22) Filed: Sep. 17, 2020

(51) Int. Cl. G01N 33/68

(52) U.S. Cl. CPC G01N 33/6863; G01N 33/6866; G01N 33/6869; G01N 2800/36; G01N 2333/52; G01N 2333/57; G01N 2333/525; G01N 2333/55; G01N 2333/5406; G01N 2333/5428; G01N 2333/5434; G01N 2333/5437; G01N 2333/54; G01N 2333/545

(57) ABSTRACT

The subject invention pertains to systems and methods diagnosing, monitoring and treating women for successful embryo implantation and establishment of pregnancy using peripheral blood cytokine profiling and administration of immune-modulators to establish an implantation-promoting microenvironment in the uterus.

### BACKGROUND OF THE INVENTION

Successful implantation of embryo is the first step of pregnancy. After fertilization, the embryo will move to uterus and implant into endometrium (implantation, day 3-9 after conception or embryo transfer, ET). A pregnancy test will be positive when the placenta starts to develop and human chorionic gonadotropin increases in urine and blood (biochemical pregnancy, at least day 9 after conception or ET) and then gestational sac and/or fetal heart pulsation can be detected by ultrasound (clinical pregnancy, at least 3 weeks after conception or ET). Currently there is no biomarker which could monitor the process and predict the success of embryo implantation (day 3-9) prior to the biochemical and clinical pregnancy (day 9 and thereafter).

The maternal immune system adapts promptly and dynamically to facilitate the implantation of the embryo. A balance between pro- and anti-inflammatory responses may play a key role in the immunomodulation to prepare for a successful implantation and prevent rejection of the implanting semi-allograft embryo in order to establish a successful pregnancy (1, 2). Immunomodulation is achieved by a complex interplay between various immune cells and cytokines at the fetal-maternal interface, among which the key-players are interleukine-10 (IL-10) and transforming growth factor-β1 (TGF-β1), although a number of other cytokines are also involved (3-5). It is recognized that different cytokines may play different roles at the varying stages of the pregnancy (6, 7). Previous animal studies and measurements of cytokines in human implantation models involving embryo and endometrium co-cultures have suggested that the initial stage of implantation is characterized by pro-inflammatory, rather than anti-inflammatory changes (8, 9). Higher concentrations of IL-1β and tumor necrosis factor-α (TNF-α), both pro-inflammatory cytokines, in endometrial secretions aspirated immediately prior to ET were associated with clinical pregnancy (10).

An in vivo study of how cytokine profiles change at the fetal-maternal interface at different stages of normal pregnancy in humans is difficult to conduct owing to ethical restrictions. Thus, some investigators have studied the changing levels of cytokines in the peripheral blood during pregnancy (11-13). It was found that early on in the pregnancy, there was a significant increase of anti-inflammatory cytokines IL-10 and TGF-β1 but a decrease of pro-inflammatory cytokines IFN-γ (14-16). The dominance of anti-inflammatory cytokines continued throughout the first two trimester of pregnancy (17, 18). However, relatively little was reported about exactly how the peripheral cytokine profile changed during the period of embryo implantation, that is, between the arrival of the embryo to the uterine cavity and confirmation of pregnancy.

The instant inventors recently reported a transient suppression of the expression of Tim-3, a regulatory molecule which suppresses immune responses, in peripheral NK (pNK) cells 3 days after ET in women with successful pregnancy (19), suggesting that the transient reduction of the Tim-3 might be associated with a brief pro-inflammatory response.

Currently, biochemical pregnancy tests and clinical ultrasound examinations are used to confirm pregnancy, but no implantation test is available to determine the success of implantation prior to the establishment of pregnancy. Such test would be highly valuable for patients and physicians to assess and potentially treat immune-related conditions that negatively interfere with the process of implantation.

### BRIEF SUMMARY OF THE INVENTION

In our study, distinct profiles and significant ratios of the serum pro- to anti-inflammatory cytokines on precise and serial time points provides a novel measurement to determine success of embryo implantation or not. It can be used as a new implantation test few days earlier than the classical biochemical and clinical pregnancy tests. Early detection of successful implantation can relief psychological stress for conception, while early identification of potentially unsuccessful implantation may allow early intervention to improve the fertility outcomes.

### BRIEF DESCRIPTION OF THE DRAWINGS

FIG. 1A shows pro-inflammatory IFN-γ cytokine blood levels of women who successfully established pregnancy compared to women who did not following embryo transfer (ET).

FIG. 1B shows pro-inflammatory IL-17 cytokine blood levels of women who successfully established pregnancy compared to women who did not following embryo transfer (ET).

FIG. 1C shows pro-inflammatory IL-12 cytokine blood levels of women who successfully established pregnancy compared to women who did not following embryo transfer (ET).

FIG. 1D shows pro-inflammatory IL-1β cytokine blood levels of women who successfully established pregnancy compared to women who did not following embryo transfer (ET).

FIG. 1E shows pro-inflammatory IL-2 cytokine blood levels of women who successfully established pregnancy compared to women who did not following embryo transfer (ET).

FIG. 1F shows pro-inflammatory IL-18 cytokine blood levels of women who successfully established pregnancy compared to women who did not following embryo transfer (ET).

FIG. 1A shows pro-inflammatory TNF-α cytokine blood levels of women who successfully established pregnancy compared to women who did not following embryo transfer (ET).

FIG. 2A shows anti-inflammatory TGF-β1 cytokine blood levels of women who successfully established pregnancy compared to women who did not following embryo transfer (ET).

FIG. 2B shows anti-inflammatory IL-10 cytokine blood levels of women who successfully established pregnancy compared to women who did not following embryo transfer (ET).

FIG. 2C shows anti-inflammatory IL-4 cytokine blood levels of women who successfully established pregnancy compared to women who did not following embryo transfer (ET).

FIG. 2D shows anti-inflammatory IL-13 cytokine blood levels of women who successfully established pregnancy compared to women who did not following embryo transfer (ET).

FIG. 2E shows anti-inflammatory IL-22 cytokine blood levels of women who successfully established pregnancy compared to women who did not following embryo transfer (ET).

FIG. 3A shows the ratio of pro-inflammatory cytokine TNF-α to anti-inflammatory cytokine IL-10 in the blood of women who successfully established pregnancy compared to women who did not following embryo transfer (ET). FIG. 3B shows the ratio of pro-inflammatory cytokine IL-1β to anti-inflammatory cytokine IL-10. FIG. 3C shows the ratio of pro-inflammatory cytokine IL-17 to anti-inflammatory cytokine IL-10. FIG. 3D shows the ratio of pro-inflammatory cytokine IFN-γ to anti-inflammatory cytokine IL-10. FIG. 3E shows the ratio of pro-inflammatory cytokine IL-12 to anti-inflammatory cytokine IL-10. FIG. 3F shows the ratio of pro-inflammatory cytokine TNF-α to anti-inflammatory cytokine TGF-β1. FIG. 3G shows the ratio of pro-inflammatory cytokine IL-1β to anti-inflammatory cytokine TGF-β1. FIG. 3H shows the ratio of pro-inflammatory cytokine IL-17 to anti-inflammatory cytokine TGF-β1. FIG. 3I shows the ratio of pro-inflammatory cytokine IFN-γ to anti-inflammatory cytokine TGF-β1. FIG. 3J shows the ratio of pro-inflammatory cytokine IL-12 to anti-inflammatory cytokine TGF-β1.

FIG. 4A shows the deviation from the baseline of the ratio of pro-inflammatory cytokine TNF-α to anti-inflammatory cytokine IL-10 in the blood of women who successfully established pregnancy compared to women who did not following embryo transfer (ET). FIG. 4B shows the deviation from the baseline of the ratio of pro-inflammatory cytokine IL-1β to anti-inflammatory cytokine IL-10. FIG. 4C shows the deviation from the baseline of the ratio of pro-inflammatory cytokine IL-17 to anti-inflammatory cytokine IL-10. FIG. 4D shows the deviation from the baseline of the ratio of pro-inflammatory cytokine IFN-γ to anti-inflammatory cytokine IL-10. FIG. 4E shows the deviation from the baseline of the ratio of pro-inflammatory cytokine IL-12 to anti-inflammatory cytokine IL-10. FIG. 4F shows the deviation from the baseline of the ratio of pro-inflammatory cytokine TNF-α to anti-inflammatory cytokine TGF-β1. FIG. 4G shows the deviation from the baseline of the ratio of pro-inflammatory cytokine IL-1β to anti-inflammatory cytokine TGF-β1. FIG. 4H shows the deviation from the baseline of the ratio of pro-inflammatory cytokine IL-17 to anti-inflammatory cytokine TGF-β1. FIG. 4I shows the deviation from the baseline of the ratio of pro-inflammatory cytokine IFN-γ to anti-inflammatory cytokine TGF-β1. FIG. 4J shows the deviation from the baseline of the ratio of pro-inflammatory cytokine IL-12 to anti-inflammatory cytokine TGF-β1.

### DETAILED DISCLOSURE OF THE INVENTION

Provided are systems and methods for the diagnosis, monitoring and treatment of women for successful embryo implantation and establishment of pregnancy using peripheral blood cytokine profiling and administration of immune-modulators for establishing an implantation-promoting uterine microenvironment.

In some embodiments of the invention, the systems and methods comprise obtaining blood samples from women undergoing blastocyst transfer during an In Vitro Fertilization (IVF) procedure. In some embodiments, the systems and methods comprise obtaining blood samples from women undergoing in utero semination. In some embodiments, the systems and methods comprise obtaining blood samples from women conceiving naturally.

In preferred embodiments, the systems and methods of the invention provide a method of diagnosing embryo implantation after embryo transfer.

In other preferred embodiments, the systems and methods of the invention provide a method of diagnosing embryo implantation after in utero semination.

In further preferred embodiments, the system sand methods of the invention provide a method of diagnosing embryo implantation after natural conception.

In some embodiments, the methods comprise obtaining a blood sample of a subject prior to embryo transfer or in utero semination; obtaining at least one first blood sample of a subject 1 to 2 days after embryo transfer or in utero semination; and/or obtaining at least one second blood sample 3 to 9 days after receiving an embryo transfer or in utero semination.

In some embodiments, the methods comprise obtaining a blood sample of a subject prior to natural conception; obtaining at least one first blood sample of a subject 1 to 5 days after natural conception; and/or obtaining at least one second blood sample 3 to 9 days after receiving a natural conception.

In some embodiments, blood samples are obtained from about 20 days prior to about 60 after an embryo transfer, in utero semination procedure, and/or natural conception. In further embodiments, blood samples are obtained from about 18 days prior to about 58 days post embryo transfer, in utero semination, and/or natural conception; from about 16 days prior to about 56 days post; from about 14 days prior to about 54 days post; from about 12 days prior to about 52 days post; from about 10 days prior to about 50 days post; from about 9 days prior to about 48 days post; from about 8 days prior to about 46 days post; from about 7 days prior to about 44 days post; from about 7 days prior to about 42 days post; from about 7 days prior to about 40 days post; from about 7 days prior to about 38 days post; from about 7 days prior to about 36 days post; from about 7 days prior to about 34 days post; from about 7 days prior to about 32 days post; from about 7 days prior to about 30 days post; from about 7 days prior to about 28 days post; from about 7 days prior to about 26 days post; from about 7 days prior to about 24 days post; from about 7 days prior to about 22 days post; from about 7 days prior to about 20 days post; from about 7 days prior to about 18 days post; from about 7 days prior to about 16 days post; from about 7 days prior to about 14 days post; from about 7 days prior to about 12 days post; from about 7 days prior to about 10 days post; from about 7 days prior to about 9 days post; from about 7 days prior to about 8 days post; from about 7 days prior to about 7 days post; from about 6 days prior to about 6 days post; from about 5 days prior to about 5 days post; from about 4 days prior to about 4 days post; from about 3 days prior to about 3 days post; from about 2 days prior to about 2 days post; from about 1 day prior to about 1 day post; and/or on the day of embryo transfer, in utero semination, and/or natural conception prior to and post embryo transfer, in utero semination, and/or natural conception.

In preferred embodiments, the methods comprise obtaining a blood sample of a subject prior to embryo transfer or in utero semination; obtaining at least one first blood sample of a subject 1 to 2 days after embryo transfer or in utero semination; and/or obtaining at least one second blood sample 3 to 9 days after receiving an embryo transfer or in utero semination.

In further embodiments, the methods comprise measuring cytokine concentrations in the blood samples. In some embodiments, the concentrations of cytokines are measured, which cytokines include, but are not limited to, IFN-γ, IL-17, IL-12, IL-1β, IL-2, IL-18, TNF-α, TGF-β1, IL-10, IL-4, IL-13, IL-22 or related molecules are measured.

In preferred embodiments, the cytokines measured include, but not limited to, IFN-γ, IL-17, IL-12, IL-10, and TGF-β1.

In some embodiments, ratios of cytokine concentrations measured in the blood samples are calculated. In preferred embodiments, the ratios of cytokine concentrations include, but are not limited to TNF-α/IL-10, IL-1β/IL-10, IL-17/IL-10, IFN-γ/IL-10, IL-12/IL-10, TNF-α/TGF-β1, IL-1β/TGF-β1, IL-17/TGF-β1, IFN-γ/TGF-β1, and IL-12/TGF-β1.

In some embodiments, the cytokines measured in the blood sample include, but are not limited to, IL-1α, IL-1β, IL-1RA, IL-18, IL-2, Il-4, IL-7, IL-9, IL-13, IL-15, IL-3, IL-5, CSF-2, CSF-3, IL-6, IL-11, IL-12, LIF, OSM, IL-10, IL-20, IL-14, IL-16, IL-17, IL-21, IL-22, IL-23, IL-35, IFN-α, IFN-β, IFN-γ, TNF-α, TNF-β, CD154, LT-β, 4-1BBL, TALL-2, CD27L, CD30L, FasL, GITRL, LIGHT, OX40L, TALL-1, Apo2L, Apo3L, OPGL, TGF-β1, TGF-β0, TGF-β3, Epo, Tpo, Flt-3L, SCF, CSF-1, MSP, XCL1, SCL2, CCL1, CCL2, CCL3, CCL4, CCL5, CCL7, CCL8, CCL11, CCL13, CCL14, CCL15, CCL15, CCL16, CCL17, CCL18, CCL19, CCL20, CCL21, CCL22, CCL23, CCL24, CCL25, CCL26, CCL27, CXCL1, CXCL2, CXCL3, CXCL4, CXCL5, CXCL6, CXCL7, CXCL8, CXCL9, CXCL10, CXCL11, CXCL12, CXCL13, CXCL14, and CX3CL1.

In some embodiments, the systems and methods of the invention comprise diagnosing embryo implantation, wherein embryo implantation is diagnosed based on the cytokine concentrations measured in the blood samples obtained from a subject who obtained an embryo transfer, in utero semination, or naturally conceived.

In some embodiments, successful embryo implantation is diagnosed if the cytokine concentrations in at least one first blood sample obtained after embryo transfer, in utero semination, or natural conception indicate a pro-inflammatory cytokine profile.

In preferred embodiments, successful embryo implantation is diagnosed if the cytokine concentrations in at least one first blood sample obtained 1-2 or 1-5 days after embryo transfer, in utero semination, or natural conception indicate a pro-inflammatory cytokine profile.

In further preferred embodiments, successful embryo implantation is diagnosed if the cytokine concentrations in at least one second blood sample obtained 3-9 or 5-9 days after embryo transfer, in utero semination, or natural conception indicate an anti-inflammatory cytokine profile.

In more preferred embodiments, successful embryo implantation is diagnosed if the cytokine concentrations in at least one first blood sample obtained 1-2 or 1-5 days after embryo transfer, in utero semination, or natural conception indicate a pro-inflammatory cytokine profile and the cytokine concentrations in at least one second blood sample obtained 3-9 or 5-9 days after embryo transfer, in utero semination, or natural conception indicate an anti-inflammatory cytokine profile.

In some embodiments, a pro-inflammatory cytokine profile comprises increased concentrations of cytokines including, but not limited to, IFN-γ, IL-17, IL-12, IL-1β, IL-2, IL-18, TNF-α in at least one first blood sample obtained 1-2 days after embryo transfer compared to the concentrations of the respective cytokines in a blood sample obtained prior to embryo transfer.

In some embodiments, a pro-inflammatory cytokine profile comprises increased concentrations of cytokines including, but not limited to, IFN-γ, IL-17, IL-12, IL-1β, IL-2, IL-18, TNF-α in at least one second sample obtained 3-9 days after embryo transfer compared to the concentrations of the respective cytokines in a blood sample obtained prior to embryo transfer.

In some embodiments, a pro-inflammatory cytokine profile comprising increased concentrations of cytokines including, but not limited to, IFN-γ, IL-17, IL-12, IL-1β, IL-2, IL-18, TNF-α in at least one first blood sample obtained 1-2 days after embryo transfer compared to the concentrations of the respective cytokines in a blood sample obtained prior to embryo transfer and a pro-inflammatory cytokine profile comprising increased concentrations of cytokines including, but not limited to, IFN-γ, IL-17, IL-12, IL-1β, IL-2, IL-18, TNF-α in at least one second sample obtained 3-9 days after embryo transfer compared to the concentrations of the respective cytokines in a blood sample obtained prior to embryo transfer provides a diagnosis of embryo implantation failure according to the methods and systems of the invention.

In some embodiments, a pro-inflammatory cytokine profile comprises increased concentrations of cytokines including, but not limited to, IFN-γ, IL-17, IL-12, IL-1β, IL-2, IL-18, TNF-α in at least one first blood sample obtained 1-2 days after in utero semination compared to the concentrations of the respective cytokines in a blood sample obtained prior to in utero semination.

In some embodiments, a pro-inflammatory cytokine profile comprises increased concentrations of cytokines including, but not limited to, IFN-γ, IL-17, IL-12, IL-1β, IL-2, IL-18, TNF-α in at least one second sample obtained 3-9 days after in utero semination compared to the concentrations of the respective cytokines in a blood sample obtained prior to in utero semination.

In some embodiments, a pro-inflammatory cytokine profile comprising increased concentrations of cytokines including, but not limited to, IFN-γ, IL-17, IL-12, IL-1β, IL-2, IL-18, TNF-α in at least one first blood sample obtained 1-2 days after in utero semination compared to the concentrations of the respective cytokines in a blood sample obtained prior to in utero semination and a pro-inflammatory cytokine profile comprising increased concentrations of cytokines including, but not limited to, IFN-γ, IL-17, IL-12, IL-1β, IL-2, IL-18, TNF-α in at least one second sample obtained 3-9 days after in utero semination compared to the concentrations of the respective cytokines in a blood sample obtained prior to in utero semination provides a diagnosis of embryo implantation failure according to the methods and systems of the invention.

In some embodiments, a pro-inflammatory cytokine profile comprises increased concentrations of cytokines including, but not limited to, IFN-γ, IL-17, IL-12, IL-1β, IL-2, IL-18, TNF-α in at least one first blood sample obtained 3-5 days after natural conception compared to the concentrations of the respective cytokines in a blood sample obtained prior to natural conception.

In some embodiments, a pro-inflammatory cytokine profile comprises increased concentrations of cytokines including, but not limited to, IFN-γ, IL-17, IL-12, IL-1β, IL-2, IL-18, TNF-α in at least one second sample obtained 6-9 days after natural conception compared to the concentrations of the respective cytokines in a blood sample obtained prior to natural conception.

In some embodiments, a pro-inflammatory cytokine profile comprising increased concentrations of cytokines including, but not limited to, IFN-γ, IL-17, IL-12, IL-1β, IL-2, IL-18, TNF-α in at least one first blood sample obtained 1-5 days after natural conception compared to the concentrations of the respective cytokines in a blood sample obtained prior to natural conception and a pro-inflammatory cytokine profile comprising increased concentrations of cytokines including, but not limited to, IFN-γ, IL-17, IL-12, IL-1β, IL-2, IL-18, TNF-α in at least one second sample obtained 5-9 days after natural conception compared to the concentrations of the respective cytokines in a blood sample obtained prior to natural conception provides a diagnosis of embryo implantation failure according to the methods and systems of the invention

In some embodiments, an anti-inflammatory cytokine profile comprises increased concentrations of cytokines including, but not limited to, TGF-β1, IL-10, IL-4, IL-13, or IL-22 in at least one first blood sample obtained 1-2 days after embryo transfer compared to the concentrations of the respective cytokines in a blood sample obtained prior to embryo transfer.

In some embodiments, an anti-inflammatory cytokine profile comprises increased concentrations of cytokines including, but not limited to, TGF-β1, IL-10, IL-4, IL-13, or IL-22 in at least one second sample obtained 3-9 days after embryo transfer compared to the concentrations of the respective cytokines in a blood sample obtained prior to embryo transfer.

In some embodiments, an anti-inflammatory cytokine profile comprises increased concentrations of cytokines including, but not limited to, TGF-β1, IL-10, IL-4, IL-13, or IL-22 in at least one first blood sample obtained 1-2 days after in utero semination compared to the concentrations of the respective cytokines in a blood sample obtained prior to in utero semination.

In some embodiments, an anti-inflammatory cytokine profile comprises increased concentrations of cytokines including, but not limited to, TGF-β1, IL-10, IL-4, IL-13, or IL-22 in at least one second sample obtained 3-9 days after in utero semination compared to the concentrations of the respective cytokines in a blood sample obtained prior to in utero semination.

In some embodiments, an anti-inflammatory cytokine profile comprises increased concentrations of cytokines including, but not limited to, TGF-β1, IL-10, IL-4, IL-13, or IL-22 in at least one first blood sample obtained 3-5 days after natural conception compared to the concentrations of the respective cytokines in a blood sample obtained prior to natural conception.

In some embodiments, an anti-inflammatory cytokine profile comprises increased concentrations of cytokines including, but not limited to, TGF-β1, IL-10, IL-4, IL-13, or IL-22 in at least one second sample obtained 6-9 days after natural conception compared to the concentrations of the respective cytokines in a blood sample obtained prior to natural conception.

In preferred embodiments, successful embryo implantation is diagnosed if the cytokine concentrations in at least one first blood sample obtained after embryo transfer, in utero semination, or natural conception indicate a pro-inflammatory cytokine profile.

In further preferred embodiments, successful embryo implantation is diagnosed if the cytokine concentrations in at least one second blood sample obtained 3-9 or 5-9 days after embryo transfer, in utero semination, or natural conception indicate an anti-inflammatory cytokine profile.

In more preferred embodiments, successful embryo implantation is diagnosed if the cytokine concentrations in at least one first blood sample obtained 1-2 days after embryo transfer or in utero semination indicate a pro-inflammatory cytokine profile and the cytokine concentrations in at least one second blood sample obtained 3-9 days after embryo transfer or in utero semination indicate an anti-inflammatory cytokine profile.

In further preferred embodiments, successful embryo implantation is diagnosed if the cytokine concentrations in at least one first blood sample obtained 3-5 days after natural conception indicate a pro-inflammatory cytokine profile and the cytokine concentrations in at least one second blood sample obtained 5-9 days after natural conception indicate an anti-inflammatory cytokine profile.

In preferred embodiments, the methods and systems of the invention calculate ratios of cytokine concentrations. In more preferred embodiments, the cytokine concentration ratios calculated include, but are not limited to, TNF-α/IL-10, IL-1β/IL-10, IL-17/IL-10, IFN-γ/IL-10, IL-12/IL-10, TNF-α/TGF-β1, IL-1β/TGF-β1, IL-17/TGF-β1, IFN-γ/TGF-β1, IL-12/TGF-β1 concentration ratios. In most preferred embodiments, the ratios of cytokine concentrations are selected from TNF-α/IL-10, IL-1β/IL-10, IFN-γ/IL-10, IL-12/IL-10, TNF-α/TGF-β1, IL-17/TGF-β1 concentration ratios.

In some embodiments, a pro-inflammatory cytokine profile comprises an increased ratio of cytokine concentrations including, but not limited to, an increased IL-1β/TGF-β1, IFN-γ/IL-10, TNF-α/IL-10, IL-1β/IL-10, IL-17/IL-10, IL-12/IL-10, and/or TNF-α/TGF-β1 ratio.

In some embodiments, a pro-inflammatory cytokine profile comprises increased ratios of cytokine concentrations including, but not limited to, IL-1β/TGF-β1, IFN-γ/IL-10, TNF-α/IL-10, IL-1β/IL-10, IL-17/IL-10, IL-12/IL-10, and/or TNF-α/TGF-β1 ratios in at least one first blood sample obtained 1-2 days after embryo transfer compared to the ratios of the respective cytokine concentrations in a blood sample obtained prior to embryo transfer.

In some embodiments, a pro-inflammatory cytokine profile comprises increased ratios of cytokine concentrations including, but not limited to, IL-1β/TGF-β1, IFN-γ/IL-10, TNF-α/IL-10, IL-1β/IL-10, IL-17/IL-10, IL-12/IL-10, and/or TNF-α/TGF-β1 ratios in at least one second blood sample obtained 3-9 days after embryo transfer compared to the ratios of the respective cytokine concentrations in a blood sample obtained prior to embryo transfer.

In some embodiments, a pro-inflammatory cytokine profile comprises increased ratios of cytokine concentrations including, but not limited to, IL-1β/TGF-β1, IFN-γ/IL-10, TNF-α/IL-10, IL-1β/IL-10, IL-17/IL-10, IL-12/IL-10, and/or TNF-α/TGF-β1 ratios in at least one first blood sample obtained 1-2 days after in utero semination compared to the ratios of the respective cytokine concentrations in a blood sample obtained prior to in utero semination.

In some embodiments, a pro-inflammatory cytokine profile comprises increased ratios of cytokine concentrations including, but not limited to, IL-1β/TGF-β1, IFN-γ/IL-10, TNF-α/IL-10, IL-1β/IL-10, IL-17/IL-10, IL-12/IL-10, and/or TNF-α/TGF-β1 ratios in at least one second blood sample obtained 3-9 days after in utero semination compared to the ratios of the respective cytokine concentrations in a blood sample obtained prior to in utero semination.

In some embodiments, a pro-inflammatory cytokine profile comprises increased ratios of cytokine concentrations including, but not limited to, IL-1β/TGF-β1, IFN-γ/IL-10, TNF-α/IL-10, IL-1β/IL-10, IL-17/IL-10, IL-12/IL-10, and/or TNF-α/TGF-β1 ratios in at least one first blood sample obtained 1-5 days after natural conception compared to the ratios of the respective cytokine concentrations in a blood sample obtained prior to natural conception.

In some embodiments, a pro-inflammatory cytokine profile comprises increased ratios of cytokine concentrations including, but not limited to, IL-1β/TGF-β1, IFN-γ/IL-10, TNF-α/IL-10, IL-1β/IL-10, IL-17/IL-10, IL-12/IL-10, and/or TNF-α/TGF-β1 ratios in at least one second blood sample obtained 5-9 days after natural conception compared to the ratios of the respective cytokine concentrations in a blood sample obtained prior to natural conception.

In some embodiments, an anti-inflammatory cytokine profile comprises a decreased ratio of cytokine concentrations including, but not limited to, a decreased IL-1β/TGF-β1, IFN-γ/IL-10, TNF-α/IL-10, IL-1β/IL-10, IL-17/IL-10, IL-12/IL-10, and/or TNF-α/TGF-β1 ratio.

In some embodiments, an anti-inflammatory cytokine profile comprises decreased ratios of cytokine concentrations including, but not limited to, IL-1β/TGF-β1, IFN-γ/IL-10, TNF-α/IL-10, IL-1β/IL-10, IL-17/IL-10, IL-12/IL-10, and/or TNF-α/TGF-β1 ratios in at least one first blood sample obtained 1-2 days after embryo transfer compared to the ratios of the respective cytokine concentrations in a blood sample obtained prior to embryo transfer.

In some embodiments, an anti-inflammatory cytokine profile comprises decreased ratios of cytokine concentrations including, but not limited to, IL-1β/TGF-β1, IFN-γ/IL-10, TNF-α/IL-10, IL-1β/IL-10, IL-17/IL-10, IL-12/IL-10, and/or TNF-α/TGF-β1 ratios in at least one second blood sample obtained 3-9 days after embryo transfer compared to the ratios of the respective cytokine concentrations in a blood sample obtained prior to embryo transfer.

In some embodiments, an anti-inflammatory cytokine profile comprises decreased ratios of cytokine concentrations including, but not limited to, IL-1β/TGF-β1, IFN-γ/IL-10, TNF-α/IL-10, IL-1β/IL-10, IL-17/IL-10, IL-12/IL-10, and/or TNF-α/TGF-β1 ratios in at least one first blood sample obtained 1-2 days after in utero semination compared to the ratios of the respective cytokine concentrations in a blood sample obtained prior to in utero semination.

In some embodiments, an anti-inflammatory cytokine profile comprises decreased ratios of cytokine concentrations including, but not limited to, IL-1β/TGF-β1, IFN-γ/IL-10, TNF-α/IL-10, IL-1β/IL-10, IL-17/IL-10, IL-12/IL-10, and/or TNF-α/TGF-β1 ratios in at least one second blood sample obtained 3-9 days after in utero semination compared to the ratios of the respective cytokine concentrations in a blood sample obtained prior to in utero semination.

In some embodiments, an anti-inflammatory cytokine profile comprises decreased ratios of cytokine concentrations including, but not limited to, IL-1β/TGF-β1, IFN-γ/IL-10, TNF-α/IL-10, IL-1β/IL-10, IL-17/IL-10, IL-12/IL-10, and/or TNF-α/TGF-β1 ratios in at least one first blood sample obtained 3-5 days after natural conception compared to the ratios of the respective cytokine concentrations in a blood sample obtained prior to natural conception.

In some embodiments, an anti-inflammatory cytokine profile comprises decreased ratios of cytokine concentrations including, but not limited to, IL-1β/TGF-β1, IFN-γ/IL-10, TNF-α/IL-10, IL-1β/IL-10, IL-17/IL-10, IL-12/IL-10, and/or TNF-α/TGF-β1 ratios in at least one first blood sample obtained 5-9 days after natural conception compared to the ratios of the respective cytokine concentrations in a blood sample obtained prior to natural conception.

In preferred embodiments, successful embryo implantation is diagnosed if the cytokine concentration ratios in at least one first blood sample obtained after embryo transfer, in utero semination, or natural conception indicate a pro-inflammatory cytokine profile.

In further preferred embodiments, successful embryo implantation is diagnosed if the cytokine concentration ratios in at least one second blood sample obtained 3-9 or 5-9 days after embryo transfer, in utero semination, or natural conception indicate an anti-inflammatory cytokine profile.

In more preferred embodiments, successful embryo implantation is diagnosed if the cytokine concentration ratios in at least one first blood sample obtained 1-2 days after embryo transfer or in utero semination indicate a pro-inflammatory cytokine profile and the cytokine concentration ratios in at least one second blood sample obtained 3-9 days after embryo transfer or in utero semination indicate an anti-inflammatory cytokine profile.

In further preferred embodiments, successful embryo implantation is diagnosed if the cytokine concentration ratios in at least one first blood sample obtained 1-5 days after natural conception indicate a pro-inflammatory cytokine profile and the cytokine concentration ratios in at least one second blood sample obtained 5-9 days after natural conception indicate an anti-inflammatory cytokine profile.

Further provided are systems and methods for treating unsuccessful embryo implantation. In some embodiments, the systems and methods of the invention comprise diagnosing a failed embryo implantation in a subject based on the cytokine concentration profiles and/or cytokine concentration ratios, and treating the subject with compositions comprising cytokines, cytokine inhibitors and/or cytokine release stimulants.

In preferred embodiments, a subject diagnosed using the systems and methods of the invention with embryo implantation failure is administered at least one composition comprising at least one cytokine, at least one cytokine inhibitor and/or at least one cytokine release stimulant. Advantageously, the timely administration of said at least one composition treats a cytokine imbalance present in said subject and enables implantation of an embryo previously diagnosed of failed implantation.

In specific embodiments, the systems and methods of the invention provide a prognosis of embryo implantation failure if a pro-inflammatory cytokine profile is diagnosed in a subject in at least one first blood sample obtained shortly after embryo transfer or in utero semination and also in subsequent blood samples including at least one second blood sample obtained 3-9 days after embryo transfer or in utero semination,

In further specific embodiments, at least one first blood sample is obtained from a subject that has undergone blastocyst transfer and/or in utero semination from about 5 minutes after the embryo transfer or in utero semination to about 3 days after embryo transfer or in utero semination; or from about 10 minutes to about 2 days; about 15 minutes to about 1 day; about 20 minutes to about 20 hours; 25 minutes to about 18 hours; about 30 minutes to about 16 hours; 40 minutes to about 14 hours; 50 minutes to about 12 hours; 1 hour to about 10 hours; 90 minutes to about 8 hours; or 2 hours to about 6 hours after the embryo transfer or in utero semination.

In further specific embodiments, at least one second blood sample is obtained from a subject that has undergone blastocyst transfer and/or in utero semination from about 3 days after the embryo transfer or in utero semination to about 9 days; from about 3.5 days to about 8.5 days; from about 4 days to about 8 days; from about 4.5 days to about 7.5 days; or from about 5 days to about 7 days after embryo transfer or in utero semination.

According to the systems and methods of the invention, if a pro-inflammatory cytokine profile is measured in the at least one first and the at least one second blood sample, the subject is diagnosed with impending embryo implantation failure and treated with a composition comprising at least cytokine, at least one cytokine inhibitor and/or at least one cytokine release stimulant.

The compositions of the invention can be administered to the subject being treated by standard routes including, but not limited to, parenteral (e.g., intravenous, intraperitoneal, intradermal, subcutaneous or intramuscular), topical, transdermal, intravaginal, or intrauterine. One route may be preferred over others, which can be determined by those skilled in the art. In preferred embodiments, the compositions of the present invention are formulated for parental administration. In another embodiment, the cytokines, cytokine inhibitors and/or cytokine release stimulants and compositions of the present invention are formulated as a sustained-release formulation.

In some embodiments, the cytokines administered include, but are not limited to, IL-1α, IL-1β, IL-1RA, IL-18, IL-2, Il-4, IL-7, IL-9, IL-13, IL-15, IL-3, IL-5, CSF-2, CSF-3, IL-6, IL-11, IL-12, LIF, OSM, IL-10, IL-20, IL-14, IL-16, IL-17, IL-21, IL-22, IL-23, IL-35, IFN-α, IFN-β, IFN-γ, TNF-α, TNF-β, CD154, LT-β, 4-1BBL, TALL-2, CD27L, CD30L, FasL, GITRL, LIGHT, OX40L, TALL-1, Apo2L, Apo3L, OPGL, TGF-β1, TGF-β2, TGF-β3, Epo, Tpo, Flt-3L, SCF, CSF-1, MSP, XCL1, SCL2, CCL1, CCL2, CCL3, CCL4, CCL5, CCL7, CCL8, CCL11, CCL13, CCL14, CCL15, CCL15, CCL16, CCL17, CCL18, CCL19, CCL20, CCL21, CCL22, CCL23, CCL24, CCL25, CCL26, CCL27, CXCL1, CXCL2, CXCL3, CXCL4, CXCL5, CXCL6, CXCL7, CXCL8, CXCL9, CXCL10, CXCL11, CXCL12, CXCL13, CXCL14, and CX3CL1.

In some embodiments, the cytokine inhibitors include, but are not limited to, anti-cytokine antibodies that immuno-neutralize cytokines including but not limited to, antibodies to IL-1α, IL-1β, IL-1RA, IL-18, IL-2, Il-4, IL-7, IL-9, IL-13, IL-15, IL-3, IL-5, CSF-2, CSF-3, IL-6, IL-11, IL-12, LIF, OSM, IL-10, IL-20, IL-14, IL-16, IL-17, IL-21, IL-22, IL-23, IL-35, IFN-α, IFN-β, IFN-γ, TNF-α, TNF-β, CD154, LT-β, 4-1BBL, TALL-2, CD27L, CD30L, FasL, GITRL, LIGHT, OX40L, TALL-1, Apo2L, Apo3L, OPGL, TGF-β1, TGF-β2, TGF-β3, Epo, Tpo, Flt-3L, SCF, CSF-1, MSP, XCL1, SCL2, CCL1, CCL2, CCL3, CCL4, CCL5, CCL7, CCL8, CCL11, CCL13, CCL14, CCL15, CCL15, CCL16, CCL17, CCL18, CCL19, CCL20, CCL21, CCL22, CCL23, CCL24, CCL25, CCL26, CCL27, CXCL1, CXCL2, CXCL3, CXCL4, CXCL5, CXCL6, CXCL7, CXCL8, CXCL9, CXCL10, CXCL11, CXCL12, CXCL13, CXCL14, and CX3CL1.

In some embodiments, the cytokine inhibitors include, but are not limited to, competitive inhibitors of cytokine receptors, including but not limited to, inhibitors of receptors of, IL-1α, IL-1β, IL-1RA, IL-18, IL-2, Il-4, IL-7, IL-9, IL-13, IL-15, IL-3, IL-5, CSF-2, CSF-3, IL-6, IL-11, IL-12, LIF, OSM, IL-10, IL-20, IL-14, IL-16, IL-17, IL-21, IL-22, IL-23, IL-35, IFN-α, IFN-β, IFN-γ, TNF-α, TNF-β, CD154, LT-β, 4-1BBL, TALL-2, CD27L, CD30L, FasL, GITRL, LIGHT, OX40L, TALL-1, Apo2L, Apo3L, OPGL, TGF-β1, TGF-β2, TGF-β3, Epo, Tpo, Flt-3L, SCF, CSF-1, MSP, XCL1, SCL2, CCL1, CCL2, CCL3, CCL4, CCL5, CCL7, CCL8, CCL11, CCL13, CCL14, CCL15, CCL15, CCL16, CCL17, CCL18, CCL19, CCL20, CCL21, CCL22, CCL23, CCL24, CCL25, CCL26, CCL27, CXCL1, CXCL2, CXCL3, CXCL4, CXCL5, CXCL6, CXCL7, CXCL8, CXCL9, CXCL10, CXCL11, CXCL12, CXCL13, CXCL14, and CX3CL1.

Advantageously, the distinct serum pro- and anti-inflammatory cytokines profiles and their ratios and the measurement of a switch from pro- to anti-inflammatory cytokine profile according to the systems and methods of the invention enable the detection, monitoring and prediction of success or failure of implantation prior to the biochemical and clinical pregnancy. Further the immunotherapies of the invention targeting the specific pro- and anti-inflammatory cytokines improve implantation and prevent implantation failure.

### Materials and Methods

### Subjects

Women undergoing ET after in-vitro-fertilization (IVF) treatment at the Prince of Wales Hospital, Chinese University of Hong Kong between November 2018 and August 2019 recruited into the study.

The inclusion criteria were women (1) having a single good-quality blastocyst transferred in fresh or frozen cycles (21); (2) maternal age 20-42 years old. The exclusion criteria include: (1) hydrosalpinx; (2) structural uterine abnormalities; (3) antiphospholipid syndrome; (4) abnormal thyroid function; (5) polycystic ovarian syndrome; (6) significant medical complications; (7) recurrent miscarriage which was defined as a history of >3 consecutive miscarriages before gestational week 20, including biochemical losses (22); (8) recurrent implantation failure which was defined as failure to achieve a clinical pregnancy after transfer of at least 4 good-quality embryos in a minimum of 3 fresh or frozen cycles in a woman age<40 years (23). All patients provided informed consent, and our Institutional Review Board approved this investigation (CREC Ref: 2014.637).

A particular strength of this prospective, longitudinal cohort study was that serial measurements were obtained at precisely timed points from the day of ET to the day of pregnancy test in a group of women undergoing blastocyst transfer. Many previous studies on the peripheral inflammatory response of pregnancy involved a single time point, often not as precisely timed and usually in terms of gestational weeks rather than days. Importantly, the observations in this study were made at an early stage of embryo implantation, well before the confirmation of pregnancy. In addition, two cohorts were included in the instant studies, one cohort with successful implantation leading to ongoing pregnancy and the other cohort with failure of implantation leading to non-conception. The latter cohort served as a comparison group with which the observations in the conception cohort could be meaningfully compared. Furthermore, samples were obtained immediately prior to ET which provided baseline measurement with which any subsequent changes after ET could be compared and quantified.

In this study, homogeneity of the conception group was ensured by excluding subjects who conceived but later miscarried and included only subjects whose pregnancy later resulted in an ongoing pregnancy.

### IVF Treatment

Ovarian stimulation was initiated by human menopausal gonadotrophins (HMG) (Pergonal, Serono, Switzerland) or recombinant follicle stimulating hormone (rFSH) (Gonad-F, Serono, Switzerland). The ovulation trigger used was 10,000 units HCG (Profasi, Serono, Switzerland) administered intramuscularly when three or more leading follicles reached 16 mm or more in diameter on transvaginal ultrasound. Transvaginal oocyte retrieval was performed 36 h after hCG trigger. Luteal support was commenced in the evening of oocyte retrieval in the form of vaginal progesterone, either with 90 mg daily dose Crinone (Merck, Germany) or Endometrin (Ferring, Saint-Prex, Switzerland) 100 mg TDS. In cases of frozen-thawed ET, either natural cycle or hormonal replacement treatment cycle was used, both were monitored with endometrial thickness, ovarian activity and hormonal levels, as previously described (24). Blastocysts were thawed and transferred five days following the estimated day of ovulation or progesterone administration respectively. Women received a single blastocyst during blastocyst transfer.

### Blood Sampling

Blood was collected by venipuncture into 10-mL EDTA coated tubes starting on the day of blastocyst transfer, and repeated 3, 6 and 9 days after, respectively. Serum was separated after centrifuged 3000 rpm at 4° C. for 10 min and stored in −80° C. for later experiments.

### Confirmation of Pregnancy

Women who underwent blastocyst transfer in our center are routinely asked to have a blood sample 9 days later for serum p-hCG measurement to verify if pregnancy had occurred and have a transvaginal ultrasonography 23 days after ET to confirm viability and location of the pregnancy. For the purpose of this study, women who have a demonstrable fetal heartbeat 23 days after blastocyst transfer with at least 20 weeks gestation formed the conception group, and women who had a negative serum β-hCG (<5 mIU/L) 9 days after blastocyst transfer formed the non-conception group.

### Cytokine Measurements

The maternal serum samples were thawed and centrifuged for measurements of all cytokines at the same time. The Luminex's xMAP® technology, a multiplexed microsphere-based cytometric assay, was used to examine the presence and relative concentration of cytokines including IFN-γ, IL-1β, IL-2, IL-4, IL-10, IL-12 (P40), IL-13, IL-17, IL-18 IL-22, and TNF-α. The concentration of TGF-β1 in serum was measured by commercial ELISA Kit, because TGF-β1 in human serum is present at higher concentrations than other cytokines.

### Multiplex Bead Arrays

Pre-coated magnetic beads (Cat #HCYTA-60K for human reactivity, Merck Millipore, Billerica, Mass., USA) were used for the determination of IL-1β, IL-2, IL-4, IL-10, IL-12 (P40), IL-13, IL-17, IL-18, IL-22, IFN-γ and TNF-α by the MILLIPLEX® MAP Human Cytokine/Chemokine/Growth Factor Panel A MAGNETIC BEAD PANEL (©EMD Millipore Corporation, Billerica, Mass. 01821 USA). Cytometric bead array permitted multiplexed analysis of different cytokines in smaller quantities of serum, compared with conventional ELISA systems. Samples were measured in duplicate following the manufacturer's instructions. Assay plates were run on a Luminex 200 instrument (Luminex, Austin, Tex.). Five-parameter logistic standard curves were fit using MiraiBio MasterPlex QT software (Hitachi sensitivity Software, South San Francisco, Calif.). The observed concentration of each analyte was calculated against standard curve regression. The lower limit of the assay sensitivity was considered as the “minimum detectable concentration plus 2 standard deviations” (MinDC+2SD), as suggested by the manufacturer. The limits of detection for each assay and the intra-assay/inter-assay coefficient of variation are listed in the Table 1.

TABLE 1
The limit of detection, intra- and interassay cofficients of variation for each cytokine.

| | | Assay limit of | Intra-assay | Inter-assay |
|---|---|---|---|---|
| | | detection | coefficient of | coefficient of |
| | | (MinDC + 2SD) | variation | variation |
| Analyte | Method | (pg/mL) | (%) | (%) |
| IL-1β | Immunology Multiplex | 0.52 | <15 | <20 |
| | Assay (Merck) | | | |
| IL-2 | Immunology Multiplex | 0.46 | <15 | <20 |
| | Assay (Merck) | | | |
| IL-4 | Immunology Multiplex | 0.29 | <15 | <20 |
| | Assay (Merck) | | | |
| IL-10 | Immunology Multiplex | 1.76 | <15 | <20 |
| | Assay (Merck) | | | |
| IL-12 | Immunology Multiplex | 6.31 | <15 | <20 |
| (p40) | Assay (Merck) | | | |
| IL-13 | Immunology Multiplex | 3.92 | <15 | <20 |
| | Assay (Merck) | | | |
| IL-17 | Immunology Multiplex | 1.16 | <15 | <20 |
| | Assay (Merck) | | | |
| IL-18 | Immunology Multiplex | 0.68 | <15 | <20 |
| | Assay (Merck) | | | |
| IL-22 | Immunology Multiplex | 13.86 | <15 | <20 |
| | Assay (Merck) | | | |
| TNF-α | Immunology Multiplex | 5.75 | <15 | <20 |
| | Assay (Merck) | | | |
| IFN-γ | Immunology Multiplex | 1.42 | <15 | <20 |
| | Assay (Merck) | | | |
| TGF-β1 | Elisa (R&D) | 4.61 | 2.43 | 8.27 |

### Quantification of TGF-β1 in Serum

In order to measure the serum level of TGF-β1 serum was collected at the same time points and quantified for TGF-β1 by Human TGF-β 1 Quantikine ELISA Kit DB100B (R&D Systems, Minneapolis, USA) according to the manufacturer's protocol.

### Statistical Analysis

The distribution of data was checked by the Shapiro-Wilk test. The cytokine levels were expressed as mean±standard error of the mean (SEM) for each group of women. Paired sample t tests were used to compare the change at each of the four time points. The comparison of data between the pregnant and non-pregnant groups was made using independent sample t tests for parametric data or Mann-Whitney U test for nonparametric data where appropriate. P values<0.05 were considered statistically significant in all statistical tests. The statistical analyses were performed with the use of SPSS (Version 22; SPSS Inc., New York, N.Y., USA).

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### Example 1—Demographics

From November 2018 to August 2019, 54 subjects were recruited. Seven subjects were excluded because of biochemical pregnancy loss, miscarriage and ectopic pregnancy. In total, 47 subjects were included in the study; 27 patients conceived and achieved a gestational age of more than 20 weeks, and 20 women did not conceive after ET. The demographic details of these two groups are compared in Table 2. The mean (±SD) age of women was 35.0 (±2.9) in the pregnant group and 37.1 (±2.0) years in non-pregnant group (p<0.05). Otherwise, these was no difference in body mass index, duration of infertility, type of infertility, baseline follicle-stimulating hormone, endometrial thickness on the day of hCG trigger and the cycle type between the two groups.

TABLE 2
Demographic characteristics of women included in the study (n = 47)

| | Pregnant | Non-pregnant | P |
|---|---|---|---|
| Parameters | (n = 27) | (n = 20) | value |
| Age (years) | 35.0 ± 2.9 | 37.1 ± 2.0 | 0.013 |
| Body mass index (Kg/m²) | 23.4 ± 4.0 | 22.0 ± 2.2 | 0.296 |
| Duration of infertility (years) | 4.6 ± 3.9 | 4.8 ± 2.7 | 0.313 |
| Type of infertility | | | |
| [symbol] Primary | 13 (48.1%) | 9 (45.0%) | |
| [symbol] Secondary | 14 (51.9%) | 11 (55.0%) | 0.831 |
| Infertility diagnosis | | | |
| [symbol] Tubal factor | 7 (26.0%) | 4 (20.0%) | |
| [symbol] Male factor | 14 (51.9%) | 9 (45.0%) | |
| [symbol] Ovulation disorder | 2 (7.4%) | 2 (10.0%) | |
| [symbol] Endometriosis | 1 (3.7%) | 1 (5.0%) | |
| [symbol] Multiple factors | 1 (3.7%) | 1 (5.0%) | |
| [symbol] Unexplained | 2 (7.4%) | 3 (15.0%) | |
| Baseline FSH (IU/I) | 6.1 (5.90-12.00) | 8.4 (4.88-13.30) | 0.209 |
| Endometrial thickness | 10.4 ± 2.6 | 10.0 ± 1.6 | 0.506 |
| (mm) at time of hCG trigger | | | |
| Cycle type | | | |
| [symbol] Fresh | 3 (11.1%) | 1 (5.0%) | |
| [symbol] Frozen | 24 (88.9%) | 19 (95.0%) | 0.469 |
| [symbol] Natural | 15 (62.5%) | 10 (52.6%) | |
| [symbol] HRT | 9 (37.5%) | 9 (47.4%) | 0.515 |

Note:
Data are presented as mean ± standard deviation (SD) for parametric data, median (range) for nonparametric data, and number (%) for categorical variables.

### Example 2—Comparison Between Groups on the Day of Embryo Transfer

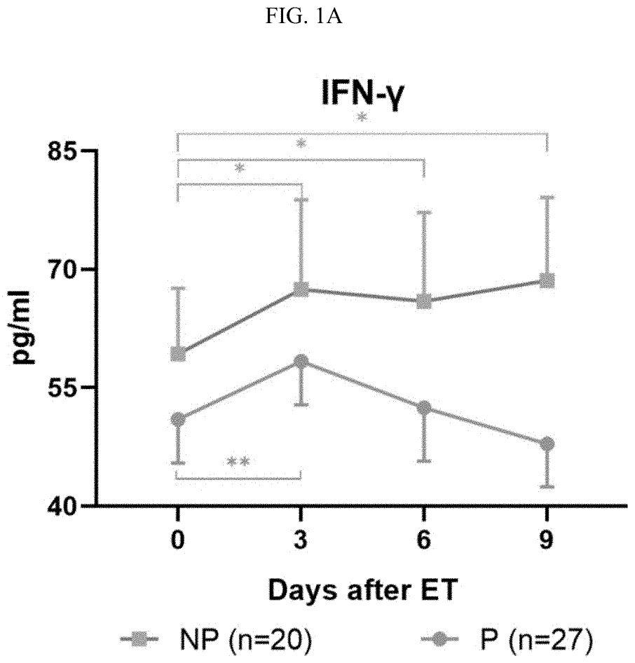

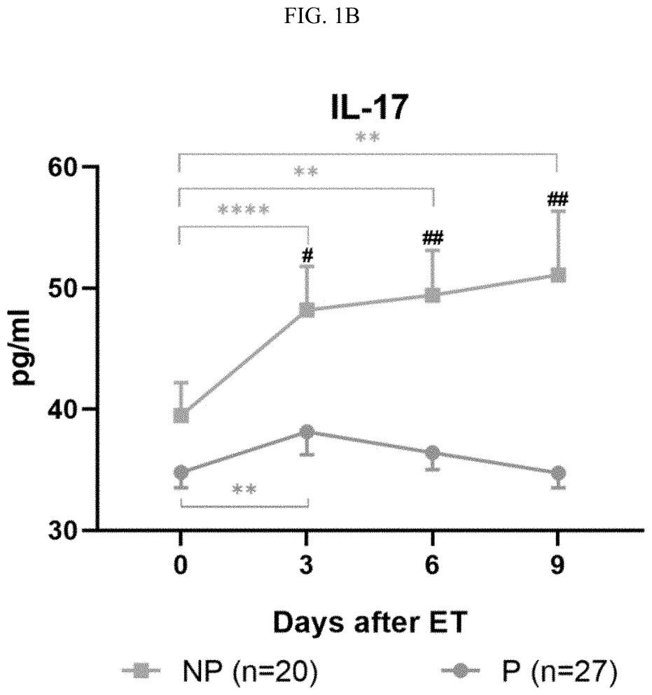

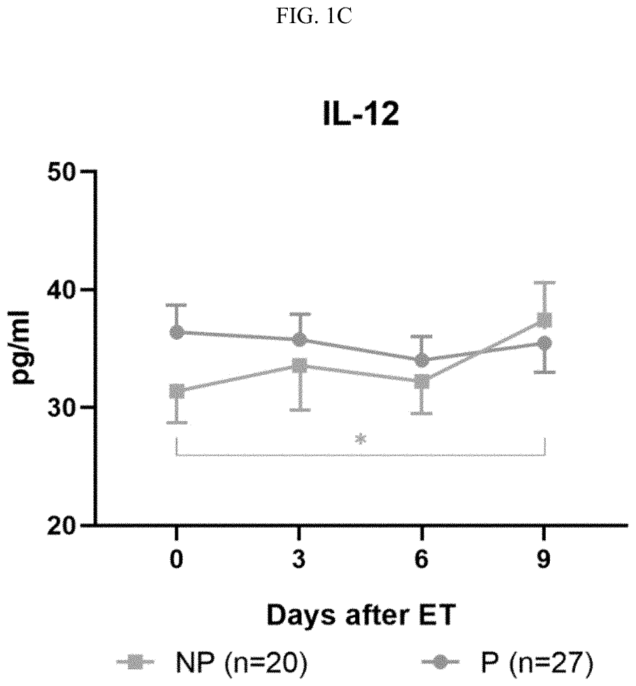

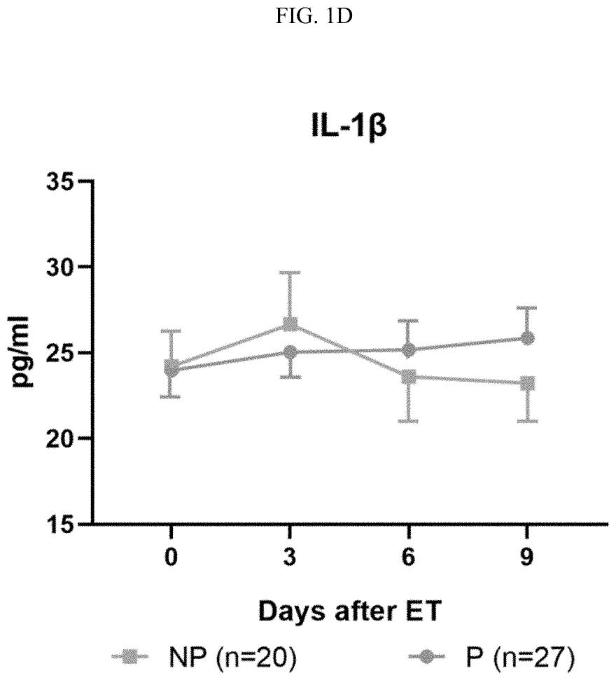

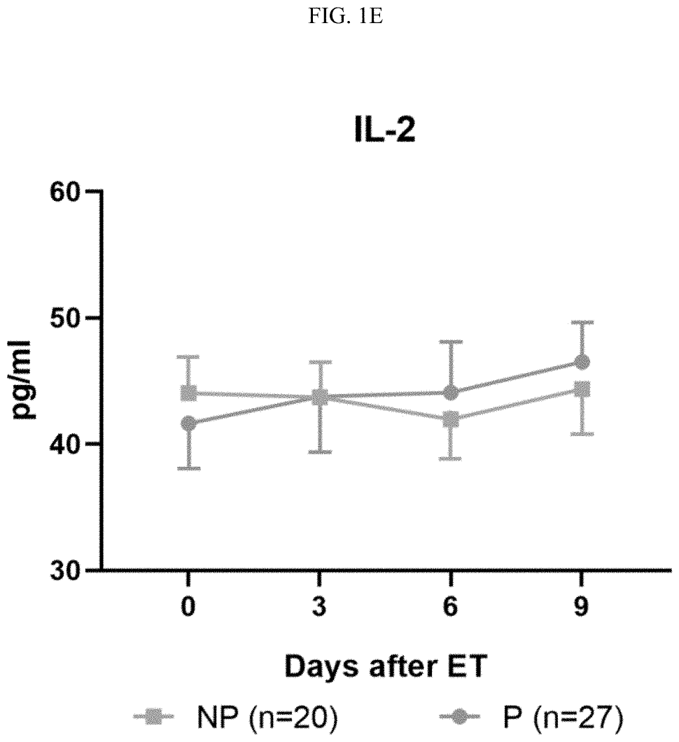

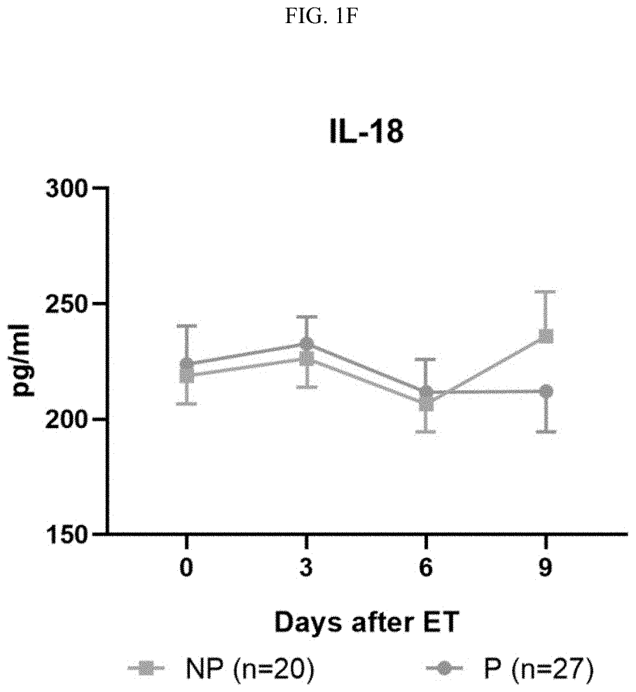

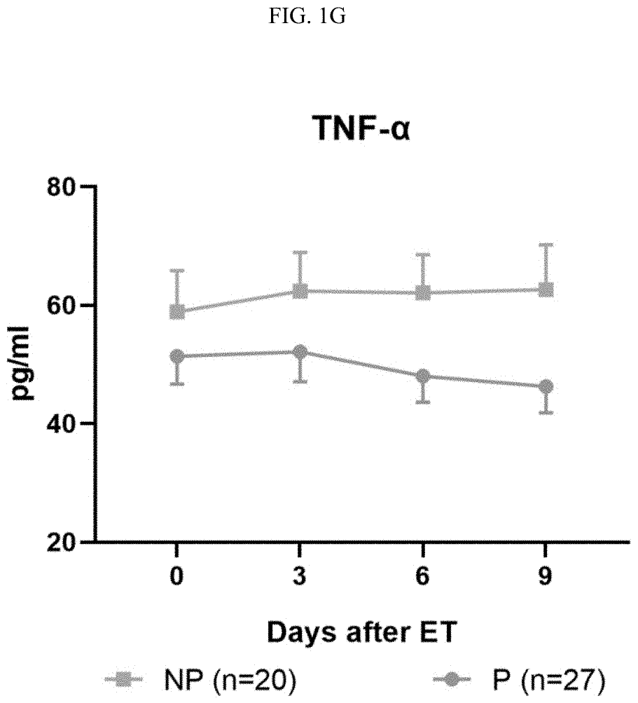

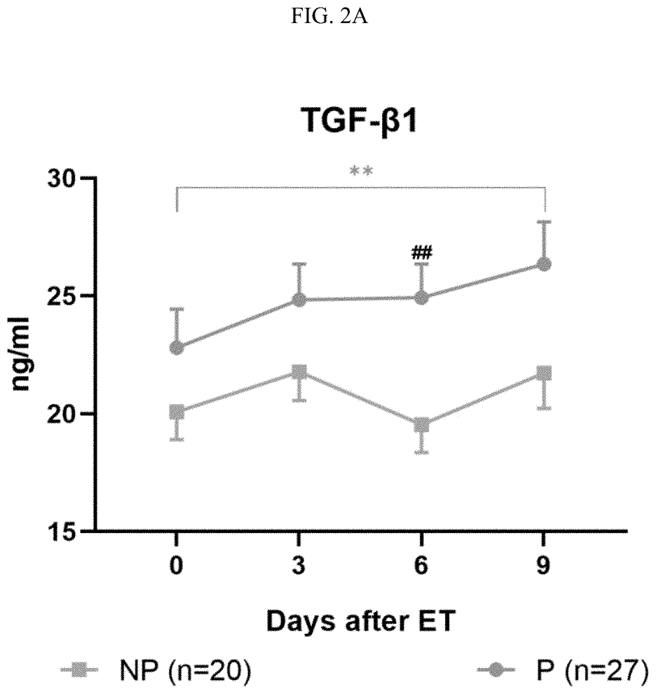

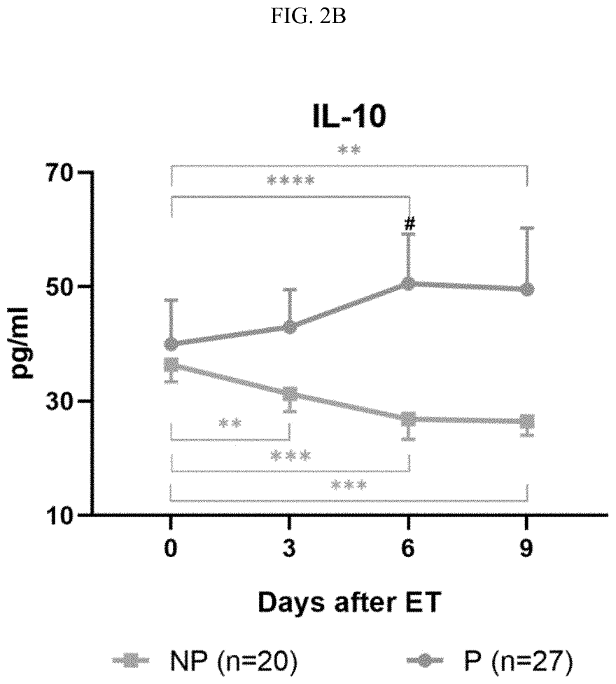

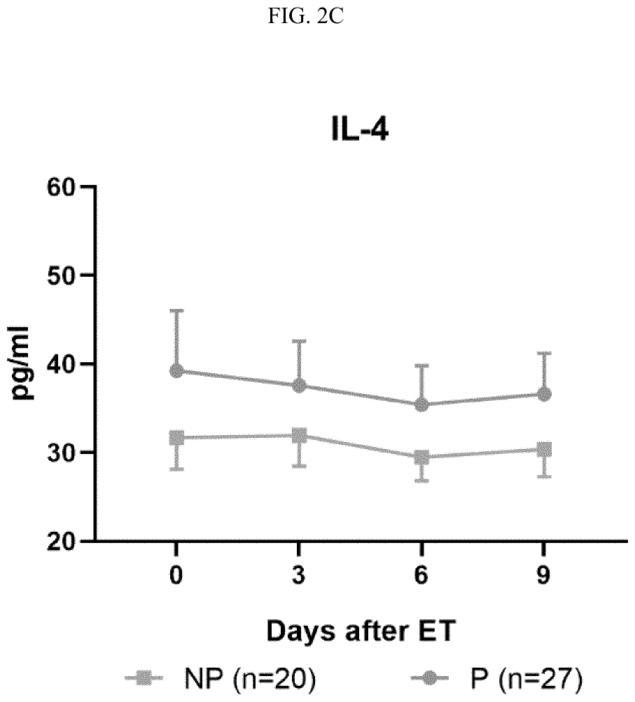

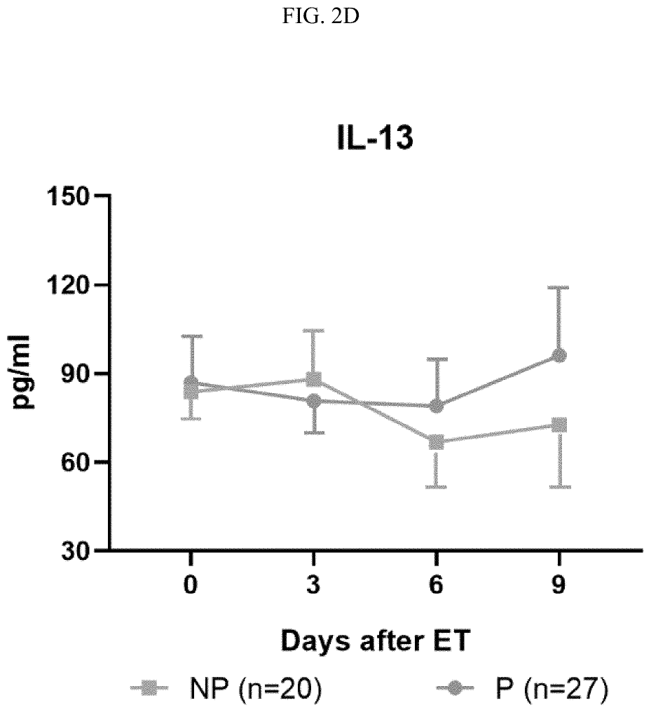

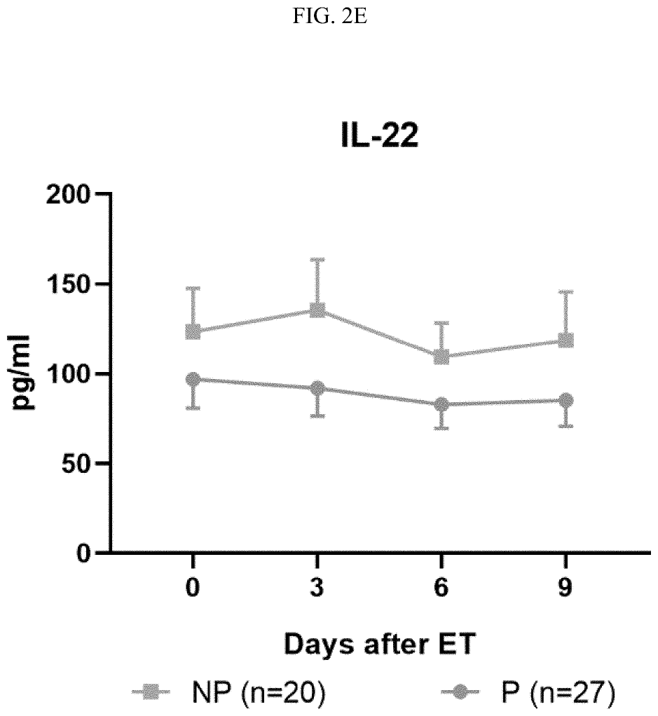

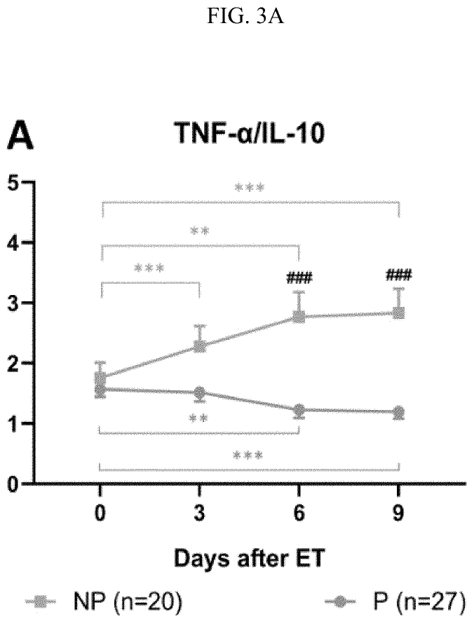

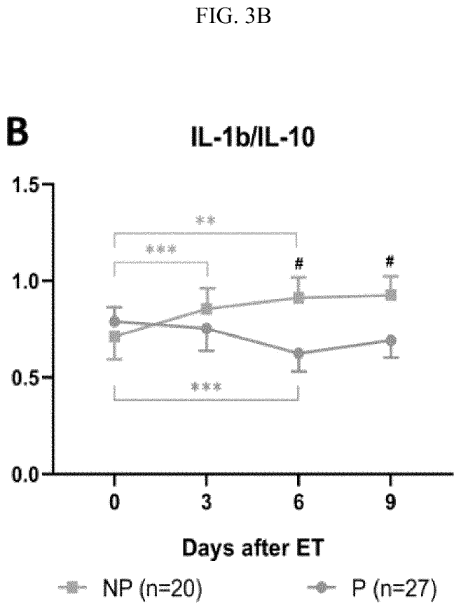

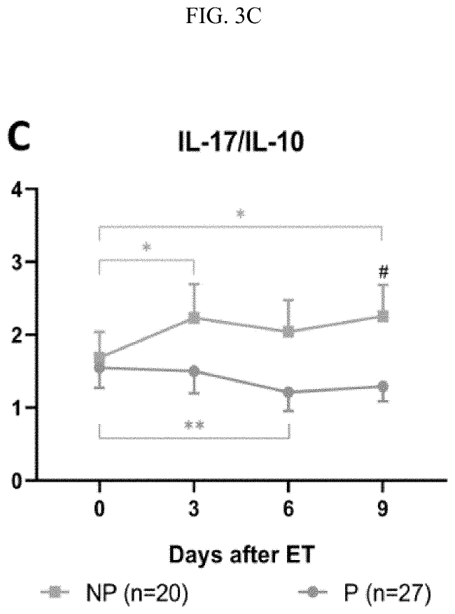

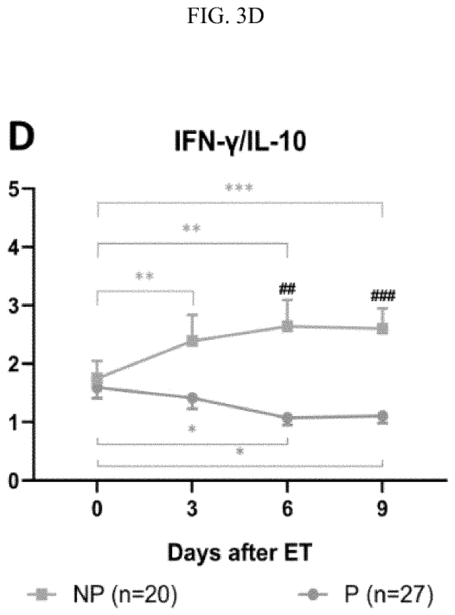

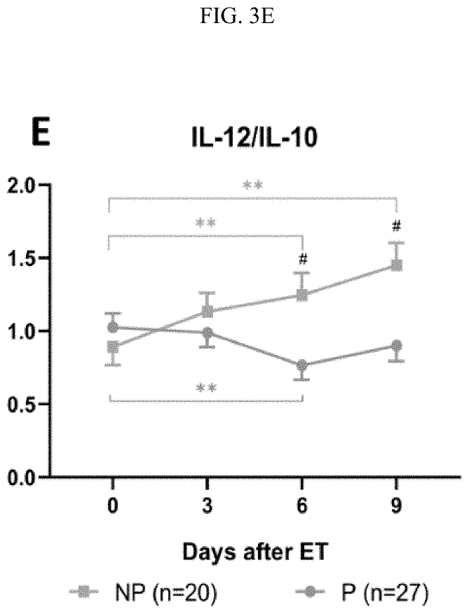

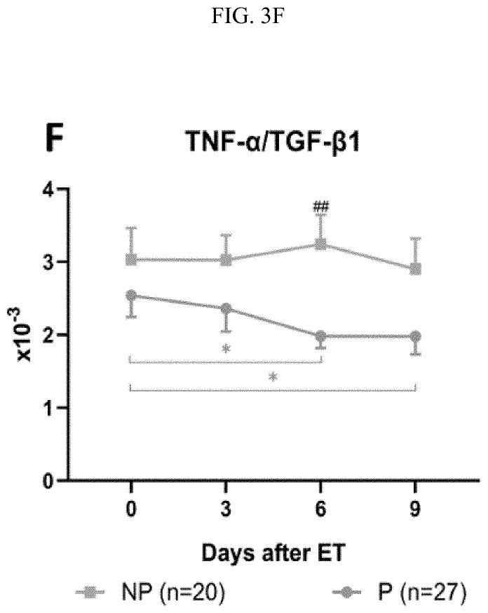

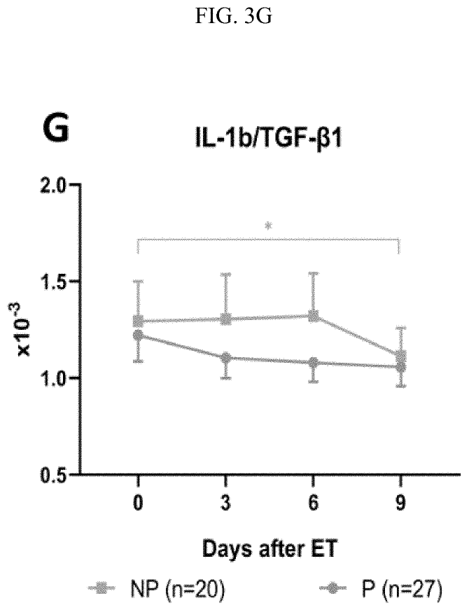

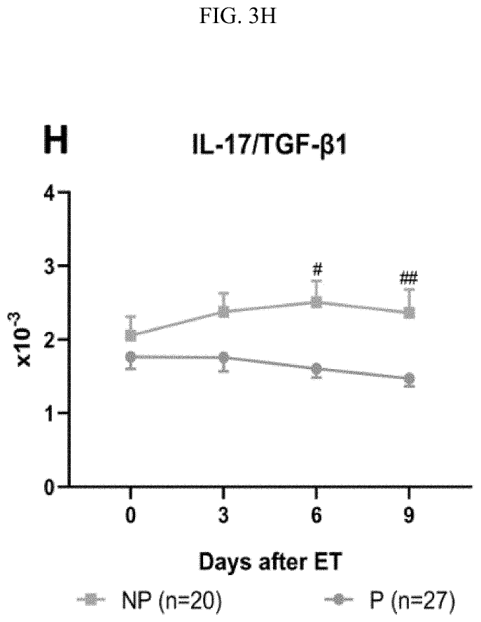

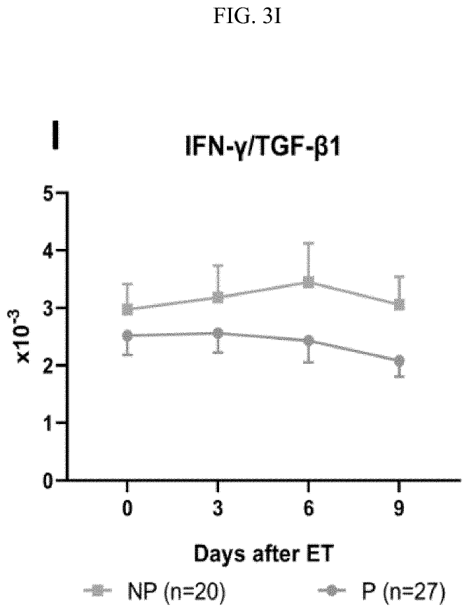

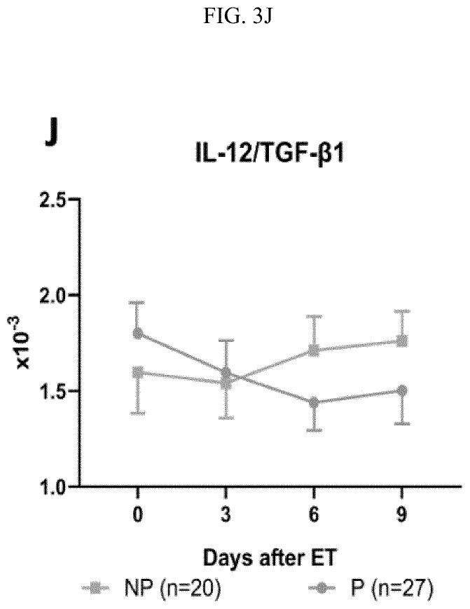

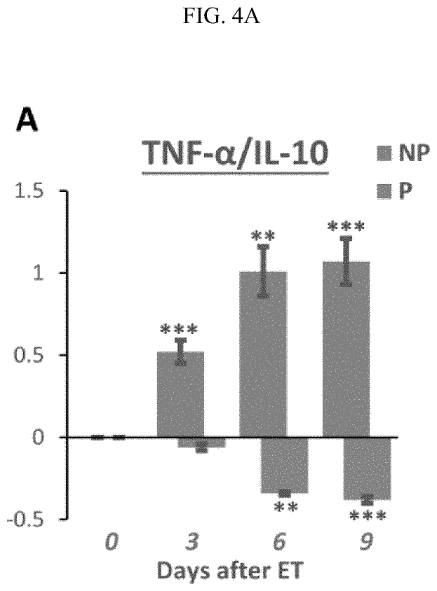

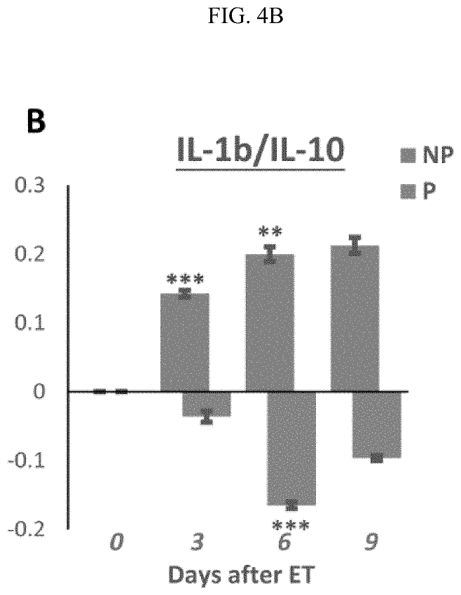

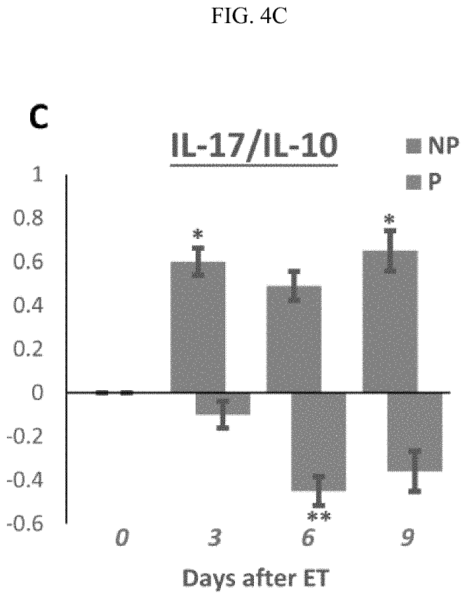

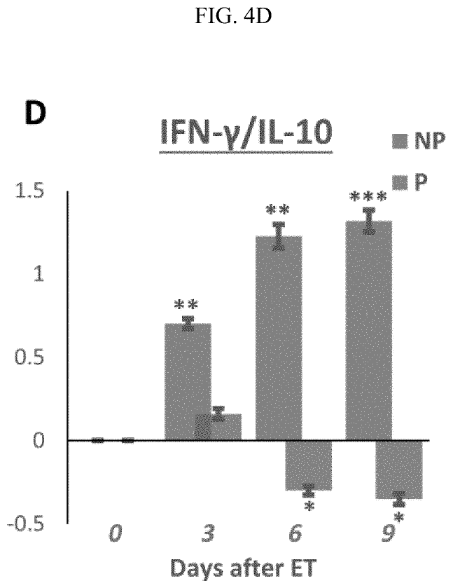

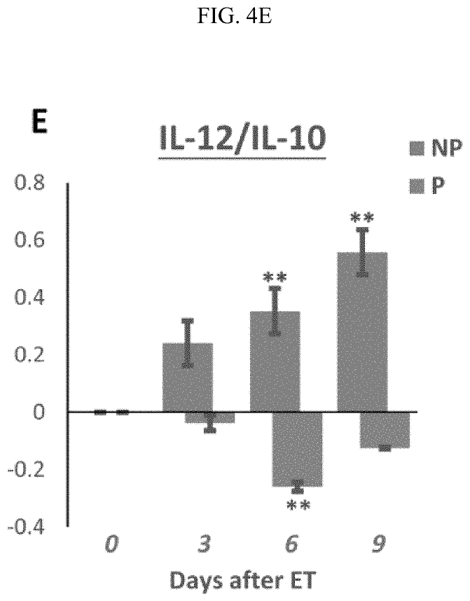

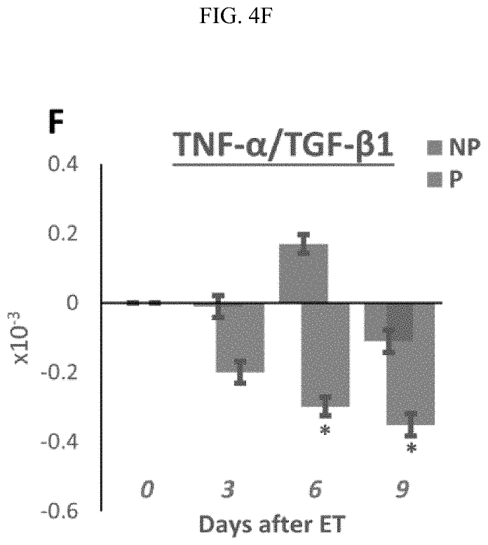

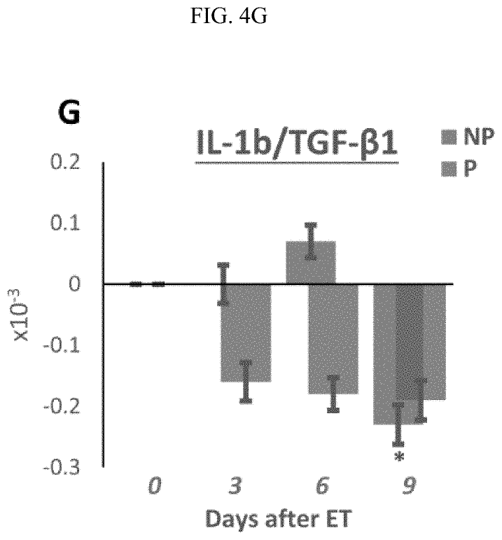

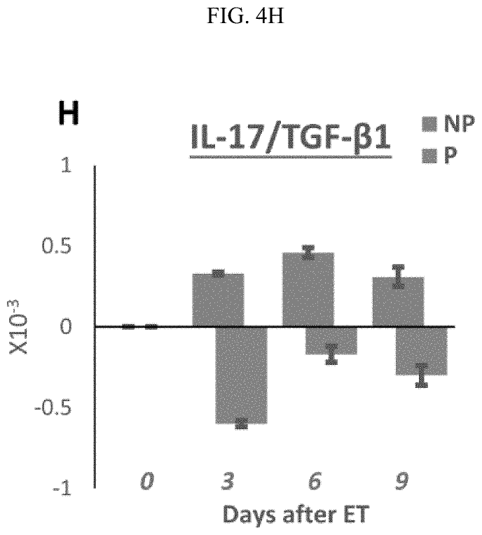

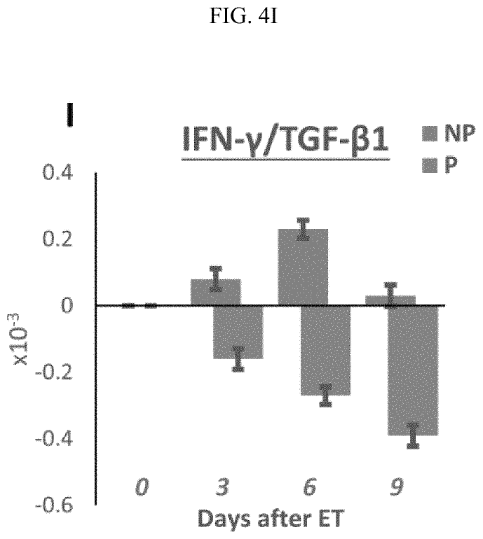

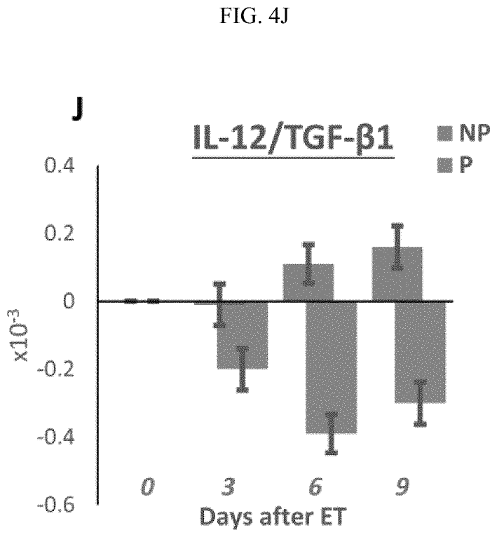

There was no significant difference in all 14 inflammation related cytokines and 10 pro-/anti-inflammation ratios between the two groups of women on the day of ET (FIGS. 1 and 2).

### Example 3—Comparison Between Groups on Day 3 after Embryo Transfer

On day 3 after ET (ET+3), the concentration of IL-17 in the pregnant group was significantly lower than that of the non-pregnant group but there was no significant difference in the remaining parameters between the two groups (FIG. 1).

### Example 4—Comparison Between Groups on Day 6 after Embryo Transfer

On day 6 after ET (ET+6), the concentration of TGF-β1 and IL-10 in the pregnant group was significantly higher, whereas the concentration of IL-17, TNF-α/IL-10, IL-1 β/IL-10, IFN-γ/IL-10, IL-12/IL-10, TNF-α/TGF-β1 and IL-17/TGF-β1 ratios in the pregnant group was significantly lower than that of the non-pregnant group; there was no significant difference in the remaining parameters between the two groups (FIG. 1).

### Example 5—Comparison Between Groups on Day 9 after Embryo Transfer

On day 9 after ET (ET+9), the concentration of IL-17, TNF-α/IL-10, 1 β/IL-10, IL-17/IL-10, IFN-γ/IL-10, IL-12/IL-10 and IL-17/TGF-β1 ratios in the pregnant group was significantly lower than that of the non-pregnant group; there was no significant difference in the remaining parameters between the two groups (FIG. 1).

### Example 6—Sequential Changes in the Non-Pregnant Group

Among women who did not conceive, when compared with the baseline, significant changes in the post-ET time points was observed for 6 parameters: (1) the concentration of IL-12 and IL-1 β/TGF-β 1 on day ET+9 was significantly higher than the baseline; (2) the concentration of IL-10 was significantly (p<0.05) lower, whereas the concentration of IFN-γ, IL-17, IFN-γ/IL-10 and TNF-α/IL-10 ratios in the non-pregnant group was significantly (p<0.05) higher than the baseline in all 3 post-ET time points (ET+3, ET+6, ET+9); (3) IL-1 β/IL-10 was significantly higher than the baseline on day ET+3 and ET+6; (4) IL-17/IL-10 was significantly increased on day ET+3 and ET+9; (4) IL-12/IL-10 was significantly elevated on day ET+6 and ET+9. Meanwhile, no significant change was observed between baseline and any of the 3 post-ET time points among the other parameters (FIGS. 1 and 2).

### Example 7—Sequential Changes in the Pregnant Group

Among women who conceived, significant changes in the post-ET time points, when compared with the baseline, was observed for 6 parameters: (1) the concentration of IL-17, IFN-γ on day ET+3 was significantly (p<0.05) higher than the baseline and the concentration of TGF-β1 on day ET+9 was significantly (p<0.05) higher than the baseline; (2) on day ET+6, IL-1 β/IL-10, IL-17/IL-10 and IL-12/IL-10 were significantly decreased than the baseline; (3) on day ET+9, the concentration of TGF-β1 was significantly higher compared to the baseline; (4) the concentration of IL-10 was significantly (p<0.05) higher, whereas the level of IFN-γ/IL-10, TNF-α/IL-10 and TNF-α/TGF-β1 ratios were significantly (p<0.05) lower than the baseline. Meanwhile, no significant change was observed between baseline and any of the 3 post-ET time points among the remaining parameters.

### Example 8—Principal Findings

In the study, we found that pregnant women who conceived after ET displayed a brief and modest increase of peripheral pro-inflammatory cytokines followed by a switch to an anti-inflammatory cytokine profile during the period of implantation. In contrast, women who failed to conceive exhibited a more pronounced pro-inflammatory cytokine change which did not manage to switch to an anti-inflammatory profile. A panel of pro- and anti-inflammatory cytokines were selected, their dynamic changes monitored and the ratios in serum of women undergoing in vitro fertilization at ET day 0 to day 9 calculated and pro- and anti-inflammatory cytokine levels and ratios compared with subsequent positive pregnancy test result (successful implantation) or with subsequent negative pregnancy test result (unsuccessful implantation). It was determined that (1) In a successful implantation group, serum pro-inflammatory cytokines IFN-γ and IL-17 were transient increased on day 3 after ET; followed by serum anti-inflammatory cytokines IL-10 and TGF-β1 increases on day 6 and/or 9 after ET. (2) The switch from increased pro-inflammatory to anti-inflammatory pattern was not found in the group of women with unsuccessful implantation. (3) The pro- and anti-inflammatory cytokine ratios of TNF-α/IL-10, IL-3/IL-10, IFN-γ/IL-10, IL-12/IL-10, TNF-α/TGF-β1 and IL-17/TGF-β1 were significantly lower in women with successful implantation than those with unsuccessful implantation as early as on day 6 after ET. This was a surprising finding in view of the fact that IL-12/IL-10, TNF-α/TGF-β1 and IL-17/TGF-β1 ratios had never been used before in pregnancy-related conditions. Therefore, a transient increase in serum pro-inflammatory cytokines followed by a switch to an increase in anti-inflammatory cytokines was identified in women with successful implantation. Advantageously, using the methods and systems of the invention differential and novel pro- and anti-inflammatory cytokine ratios between women with successful implantation and those with unsuccessful implantation before pregnancy being established were identified. Advantageously, the distinct serum pro- to anti-inflammatory cytokines profiles and their ratios can be used as biomarkers to detect, monitor and predict the success or failure of implantation prior to the biochemical and clinical pregnancy. Further provided are immunotherapies targeting the specific pro- and anti-inflammatory cytokines to improve implantation and prevent implantation failure.

### Example 9—Biomarkers and Immuno-Modulatory Treatment

The characteristic change in peripheral cytokine profile during successful implantation, well before confirmation of pregnancy, serum biomarkers to monitor implantation and to understand the mechanism of its failure, especially in women who experience recurrent implantation failure after IVF. These biomarkers can be used to detect and measure the immunological contribution to reproductive failure, to assign and administer immune-modulatory treatments to patients with biomarker profiles indicative of impending embryo implantation failure and to monitor the response to immuno-modulatory treatments administered to a subject.

### Example 10—Interpretation of the Data

The data from our study indicate that a switch from a pro-inflammatory to an anti-inflammatory environment can be detected by a significant increase of IL-10 in peripheral blood as early as 6 days after embryo transfer and that if the switch does not occur the chance of successful pregnancy outcome is reduced. The data further indicate that the switch from pro-inflammatory to anti-inflammatory peripheral blood cytokine levels may be a reflection of changes in cytokine levels in the endometrium, where by day 9 after ET the embryo has already migrated across the luminal epithelium and invaded into the stroma. Although it is challenging to measure how changes in local cytokine levels at the endometrial/embryo interface are reflected in the peripheral blood because cytokines are produced from multiple tissues and their major source in the blood are most likely circulating leucocytes, our data indicate that the levels detected in the blood reflect a systemic immunomodulation that, at least in part, driven by the hormone changes associated with early pregnancy. Importantly, both estrogen and progesterone have been shown to have immunomodulatory actions and other possible candidates, including hCG could contribute but are not yet detectable in peripheral blood at 3 to 6 days after embryo transfer.

The data of the invention indicate that the mechanism for the early peripheral cytokine switch is related to and in response to a crosstalk between the embryo and the endometrium which precedes successful implantation.

The pattern of change in cytokine levels seen in this study is similar to changes in Tim-3 seen in our previous study which showed that the expression of Tim-3 was transiently and significantly decreased on day ET+3 and started to increase on day ET+6 with further significant increase on day ET+9 in women with successful pregnancy (19). Tim-3 is a negative regulatory molecule which suppresses the production of inflammatory cytokines in peripheral NK (pNK) cells. The observations in our current study, along with the earlier findings of a similar biphasic change of Tim-3 in pNK cells indicate that Tim-3 may be involved in driving the cytokine switch in peripheral blood. The mechanism is plausible as it has been reported that a lack of Tim-3 induces increased production of IFN-γ whereas abundance of Tim-3 promotes increased production of IL-10 and TGF-β1 in pNK cells in vitro.(34).

No differences in the levels of any of the cytokines measured on the day of ET were seen in women who conceived and those who did not. This is in agreement with a previous study that showed that levels of IFN-γ, IL-4, IL-5, IL-10, IL-12, IL-13, IL-17, TNF-α and granulocyte macrophage colony-stimulating factor, GM-CSF in peripheral blood at several time points before ET and on the day of ET were not predictive of IVF outcome (35). Taken together, it appears that baseline cytokine profile, prior to ET, is unlikely to predict success or failure of implantation, but the immediate change in profile in response to ET is more informative.

Significant differences in peripheral blood cytokine levels between women who conceived and those who did not at various days after ET were seen for IFN-γ, IL-17, IL-12, IL-10 and TGF-β, while no differences were seen for the other cytokines. Although various other studies have shown that these cytokines may be involved both positively and negatively in the process of embryo implantation (36-38), the exact mechanism by which these peripheral blood changes in cytokine levels which occur immediately after ET relate to the process of implantation has not previously been determined.

The ratio TNF-α/IL-10, IL-β/IL-10, IFN-γ/IL-10, IL-12/IL-10, TNF-α/TGF-β1 and IL-17/TGF-β1 levels in peripheral blood were significantly decreased in women who conceived compared to those who did not. Many previous studies have used these ratios (or their inverse) to represent the ratios of proinflammatory to anti-inflammatory cytokines and shown that abnormal ratios are associated with reproductive failure (39, 40). A recent longitudinal study also showed that the IL-10/TNFα ratios in women undergoing IVF were higher in women with successful pregnancy outcomes compared to those who subsequently miscarried (18). However, in these prior studies samples were only obtained from week 4 of pregnancy and no measurements at the time of embryo implantation were undertaken. Since cytokines work in concert to exert physical functions, the other increased ratios in women who subsequently failed to become pregnant further verify the potential application of pro-/anti-inflammatory cytokines for monitoring and predicting embryo implantation. Furthermore, therapeutic interventions aimed at changing the cytokine microenvironment in the uterus in general and/or at the site of implantation specifically can be used to treat patients that have a cytokine profile incompatible with successful embryo implantation and successful initiation of pregnancy. Hence, the dynamic measurement of different cytokines and ratios establishes a propounding immune landscape that can be used to evaluate the progress of embryo implantation and implement treatments for improvement of embryo implantation based on cytokine adjustment therapy.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

1. Arck P C, Hecher K. Fetomaternal immune cross-talk and its consequences for maternal and offspring's health. Nature medicine 2013; 19:548-56.
2. Miko E, Meggyes M, Doba K, Barakonyi A, Szereday L. Immune Checkpoint Molecules in Reproductive Immunology. Frontiers in immunology 2019; 10:846.
3. Lidstrom C, Matthiesen L, Berg G, Sharma S, Ernerudh J, Ekerfelt C. Cytokine secretion patterns of NK cells and macrophages in early human pregnancy decidua and blood: implications for suppressor macrophages in decidua. American journal of reproductive immunology 2003; 50:444-52.
4. Plevyak M, Hanna N, Mayer S, Murphy S, Pinar H, Fast L et al. Deficiency of decidual IL-10 in first trimester missed abortion: a lack of correlation with the decidual immune cell profile. American journal of reproductive immunology 2002; 47:242-50.
5. Chang R Q, Shao J, Meng Y H, Wang J, Li D J, Li M Q. Decidual RANKL/RANK interaction promotes the residence and polarization of TGF-beta1-producing regulatory gammadelta T cells. Cell death & disease 2019; 10:113.
6. Thaxton J E, Sharma S. Interleukin-10: a multi-faceted agent of pregnancy. American journal of reproductive immunology 2010; 63:482-91.
7. Ghaebi M, Nouri M, Ghasemzadeh A, Farzadi L, Jadidi-Niaragh F, Ahmadi M et al. Immune regulatory network in successful pregnancy and reproductive failures. Biomedicine & pharmacotherapy=Biomedecine & pharmacotherapie 2017; 88:61-73.
8. Dekel N, Gnainsky Y, Granot I, Racicot K, Mor G. The role of inflammation for a successful implantation. American journal of reproductive immunology 2014; 72:141-7.
9. Mor G, Aldo P, Alvero A B. The unique immunological and microbial aspects of pregnancy. Nature reviews Immunology 2017; 17:469-82.
10. Boomsma C M, Kavelaars A, Eijkemans M J, Lentjes E G, Fauser B C, Heijnen C J et al. Endometrial secretion analysis identifies a cytokine profile predictive of pregnancy in IVF. Human reproduction 2009; 24:1427-35.
11. Martinez-Garcia E A, Chavez-Robles B, Sanchez-Hernandez P E, Nunez-Atahualpa L, Martin-Maquez B T, Munoz-Gomez A et al. IL-17 increased in the third trimester in healthy women with term labor. American journal of reproductive immunology 2011; 65:99-103.
12. Denney J M, Nelson E L, Wadhwa P D, Waters T P, Mathew L, Chung E K et al. Longitudinal modulation of immune system cytokine profile during pregnancy. Cytokine 2011; 53:170-7.
13. Wu M Y, Chen H F, Chen S U, Chao K H, Yang Y S, Ho H N. Increase in the production of interleukin-10 early after implantation is related to the success of pregnancy. American journal of reproductive immunology 2001; 46:386-92.
14. Hanna N, Hanna I, Hleb M, Wagner E, Dougherty J, Balkundi D et al. Gestational age-dependent expression of IL-10 and its receptor in human placental tissues and isolated cytotrophoblasts. Journal of immunology 2000; 164:5721-8.
15. Power L L, Popplewell E J, Holloway J A, Diaper N D, Warner J O, Jones C A. Immunoregulatory molecules during pregnancy and at birth. Journal of reproductive immunology 2002; 56:19-28.
16. Veith G L, Rice G E. Interferon gamma expression during human pregnancy and in association with labour. Gynecologic and obstetric investigation 1999; 48:163-7.
17. Singh M, Orazulike N C, Ashmore J, Konje J C. Changes in maternal serum transforming growth factor beta-1 during pregnancy: a cross-sectional study. BioMed research international 2013; 2013:318464.
18. Kaislasuo J, Simpson S, Petersen J F, Peng G, Aldo P, Lokkegaard E et al. IL-10 to TNFalpha ratios throughout early first trimester can discriminate healthy pregnancies from pregnancy losses. Am J Reprod Immunol 2020; 83:e13195.
19. Zhang T, Zhu W, Zhao Y, Cheung W C, Liu Y, Chen X et al. Early transient suppression of immune checkpoint proteins T-cell immunoglobulin mucin-3 and programmed cell death-1 in peripheral blood lymphocytes after blastocyst transfer is associated with successful implantation. Fertil Steril 2020.
21. Alpha Scientists in Reproductive M, Embryology ESIGo. The Istanbul consensus workshop on embryo assessment: proceedings of an expert meeting†. Human Reproduction 2011; 26:1270-83%@ 0268-1161.
22. Chen X, Mariee N, Jiang L, Liu Y, Wang C C, Li T C et al. Measurement of uterine natural killer cell percentage in the periimplantation endometrium from fertile women and women with recurrent reproductive failure: establishment of a reference range. Am J Obstet Gynecol 2017; 217:680 e1-e6.
23. Coughlan C, Ledger W, Wang Q, Liu F, Demirol A, Gurgan T et al. Recurrent implantation failure: definition and management. Reprod Biomed Online 2014; 28:14-38.
24. Law T S M, Cheung W C, Wu F, Zhang R, Chung J P W, Wang C C et al. Endometrial Vascularization Characterized by Optical Coherence Tomography and Immunohistochemistry in Women Undergoing In Vitro Fertilization-Embryo Transfer Treatment. Medicina (Kaunas) 2019; 55.
25. Laird S M, Tuckerman E M, Cork B A, Linjawi S, Blakemore A I, Li T C. A review of immune cells and molecules in women with recurrent miscarriage. Human reproduction update 2003; 9:163-74.
26. Inagaki N, Stem C, McBain J, Lopata A, Kornman L, Wilkinson D. Analysis of intra-uterine cytokine concentration and matrix-metalloproteinase activity in women with recurrent failed embryo transfer. Human reproduction 2003; 18:608-15.
27. Boomsma C M, Kavelaars A, Eijkemans M J, Amarouchi K, Teklenburg G, Gutknecht D et al. Cytokine profiling in endometrial secretions: a non-invasive window on endometrial receptivity. Reproductive biomedicine online 2009; 18:85-94.
28. Chaouat G, Dubanchet S, Ledée N. Cytokines: important for implantation? Journal of assisted reproduction and genetics 2007; 24:491-505%@ 1058-0468.
29. Dimitriadis E, White C A, Jones R L, Salamonsen L A. Cytokines, chemokines and growth factors in endometrium related to implantation. Human reproduction update 2005; 11:613-30%@ 1460-2369.
30. Van Mourik M S M, Macklon N S, Heijnen C J. Embryonic implantation: cytokines, adhesion molecules, and immune cells in establishing an implantation environment. Journal of leukocyte biology 2009; 85:4-19%@ 0741-5400.
31. Saito S, Nakashima A, Shima T, Ito M. Th1/Th2/Th17 and regulatory T-cell paradigm in pregnancy. American journal of reproductive immunology 2010; 63:601-10%@1046-7408.
32. Ochoa-Bernal M A, Fazleabas A T. Physiologic Events of Embryo Implantation and Decidualization in Human and Non-Human Primates. International journal of molecular sciences 2020; 21.
33. Oertelt-Prigione S. Immunology and the menstrual cycle. Autoimmunity reviews 2012; 11:A486-A92%@ 1568-9972.
34. Li Y, Zhang J, Zhang D, Hong X, Tao Y, Wang S et al. Tim-3 signaling in peripheral NK cells promotes maternal-fetal immune tolerance and alleviates pregnancy loss. Science signaling 2017; 10.
35. Persson M, Ekerfelt C, Jablonowska B, Jonsson Y, Ernerudh J, Jenmalm M C et al. Immunological status in patients undergoing in vitro fertilisation: responses to hormone treatment and relationship to outcome. Journal of reproductive immunology 2012; 96:58-67.
36. Hossein H, Mahroo M, Abbas A, Firouzeh A, Nadia H. Cytokine production by peripheral blood mononuclear cells in recurrent miscarriage. Cytokine 2004; 28:83-6.
37. Comba C, Bastu E, Dural O, Yasa C, Keskin G, Ozsurmeli M et al. Role of inflammatory mediators in patients with recurrent pregnancy loss. Fertility and sterility 2015; 104:1467-74 el.
38. Hadinedoushan H, Mirahmadian M, Aflatounian A. Increased natural killer cell cytotoxicity and IL-2 production in recurrent spontaneous abortion. American journal of reproductive immunology 2007; 58:409-14.
39. Calleja-Agius J, Schembri-Wismayer P, Calleja N, Brincat M, Spiteri D. Obstetric outcome and cytokine levels in threatened miscarriage. Gynecological Endocrinology 2011; 27:121-7%@ 0951-3590.
40. Verma P, Verma R, Nair R R, Budhwar S, Khanna A, Agrawal N R et al. Altered crosstalk of estradiol and progesterone with Myeloid-derived suppressor cells and Th1/Th2 cytokines in early miscarriage is associated with early breakdown of maternal-fetal tolerance. American Journal of Reproductive Immunology 2019; 81:e13081%@ 1046-7408.

We claim:

1. A method of diagnosing embryo implantation in a subject after embryo transfer or in utero semination, the method comprising:
obtaining a blood sample from a subject prior to embryo transfer or in utero semination;
obtaining at least one first blood sample from the subject 1 to 2 days after embryo transfer or in utero semination;
obtaining at least one second blood sample from the subject 3 to 9 days after receiving an embryo transfer or in utero semination;
measuring cytokine concentrations in the blood samples;
informing the subject of successful embryo implantation if the cytokine concentrations in the at least one first blood sample compared to the blood sample obtained prior to embryo transfer or in utero semination indicate a pro-inflammatory cytokine profile and the cytokine concentrations in the at least one second blood sample compared to the blood sample obtained prior to embryo transfer or in utero semination indicate an anti-inflammatory cytokine profile,
informing the subject of failed embryo implantation if the cytokine concentrations in the at least one first blood sample and the at least second blood sample compared to the blood sample obtained prior to embryo transfer or in utero semination indicate a pro-inflammatory cytokine profile; and
treating the subject with failed embryo implantation with a composition that induces anti-inflammatory cytokines.

2. The method according to claim 1, wherein the cytokine concentrations measured comprise IFN-γ, IL-17, IL-12, IL-1β, IL-2, IL-18, TNF-α, TGF-β1, IL-10, IL-4, IL-13, or IL-22.

3. The method according to claim 2, wherein the cytokine concentrations measured are selected from the group consisting of IFN-γ, IL-17, IL-12, IL-10, and TGF-β1.

4. The method according to claim 1 further comprising calculating ratios of cytokine concentrations.

5. The method according to claim 4, wherein the ratios of cytokine concentrations comprise TNF-α/IL-10, IL-1β/IL-10, IL-17/IL-10, IFN-γ/IL-10, IL-12/IL-10, TNF-α/TGF-β1, IL-1β/TGF-β1, IL-17/TGF-β1, IFN-γ/TGF-β1, or IL-12/TGF-β1 concentration ratios.

6. The method according to claim 5, wherein the ratios of cytokine concentrations are selected from the group consisting of TNF-α/IL-10, IL-1β/IL-10, IFN-γ/IL-10, IL-12/IL-10, TNF-α/TGF-β1, and IL-17/TGF-β1.

7. The method according to claim 1, wherein a pro-inflammatory cytokine profile is indicated when a concentration of at least one of IFN-γ, IL-17, IL-12, IL-1β, IL-2, IL-18, and TNF-α is higher in the at least one first blood sample or the at least one second blood sample compared to the blood sample obtained prior to embryo transfer or in utero semination.

8. The method according to claim 1, wherein an anti-inflammatory cytokine profile is indicated when a concentration of at least one of TGF-β1, IL-10, IL-4, IL-13, and IL-22 is higher in the at least one first blood sample or the at least one second blood sample compared to the blood sample obtained prior to embryo transfer or in utero semination.

9. A method of diagnosing embryo implantation in a subject after natural conception, the method comprising:
obtaining a blood sample from a subject prior to natural conception;
obtaining at least one first blood sample from the subject 3 to 5 days after natural conception;
obtaining at least one second blood sample from the subject 6 to 9 days after natural conception;
measuring cytokine concentrations in the blood samples;
informing the subject of successful embryo implantation if the cytokine concentrations in the at least one first blood sample compared to the blood sample obtained prior to embryo transfer indicate a pro-inflammatory cytokine profile and the cytokine concentrations in the at least one second blood sample compared to the blood sample obtained prior to embryo transfer indicate an anti-inflammatory cytokine profile; or
informing the subject of failed embryo implantation if the cytokine concentrations in the at least one first blood sample and the at least one second blood sample compared to the blood sample obtained prior to embryo transfer indicate a pro-inflammatory cytokine profile; and
treating the subject with failed embryo implantation with a composition that induces anti-inflammatory cytokines.

10. The method according to claim 9, wherein the cytokine concentrations measured comprise IFN-γ, IL-17, IL-12, IL-1β, IL-2, IL-18, TNF-α, TGF-β1, IL-10, IL-4, IL-13, or IL-22.

11. The method according to claim 9, further comprising calculating ratios of cytokine concentrations.

12. The method according to claim 11, wherein the ratios of cytokine concentrations comprise TNF-α/IL-10, IL-1β/IL-10, IL-17/IL-10, IFN-γ/IL-10, IL-12/IL-10, TNF-α/TGF-β1, IL-1β/TGF-β1, IL-17/TGF-β1, IFN-γ/TGF-β1, or IL-12/TGF-β1 concentration ratios.
